(19) United States
(12) Patent Application Publication
(10) Pub. No.: US 2021/0113586 A1
(43) Pub. Date: Apr. 22, 2021

(54) NEW USE AND METHODS OF MODULATING IMMUNE RESPONSES

(71) Applicant: DIAMYD MEDICAL AB, Stockholm (SE)

(72) Inventors: Bryndis BIRNIR, Stockholm (SE); Ulf HANNELIUS, Stockholm (SE); Anton LINDQVIST, Stockholm (SE)

(21) Appl. No.: 16/971,890

(22) PCT Filed: Feb. 25, 2019
(86) PCT No.: PCT/SE2019/050166

§ 371 Date: Aug. 21, 2020

(30) Foreign Application Priority Data
Feb. 23, 2018 (SE) 1850211-2
Mar. 12, 2018 (SE) 1850273-2

(51) Int. Cl. A61K 31/5517; A61K 31/197; A61K 31/515; A61K 31/5513; A61P 37/02; A61P 3/10; G01N 33/50

(52) U.S. Cl. CPC A61K 31/5517; A61K 31/197; A61K 31/515; A61K 31/5513; A61P 37/02; G01N 2800/7095; G01N 33/5091; G01N 2800/50; G01N 2800/52; G01N 2800/24; A61P 3/10

(57) ABSTRACT

The present invention relates to methods for identifying patients a subject at risk of developing an autoimmune or inflammatory disorder, as well as methods of prevention of development of an autoimmune or inflammatory disorder, comprising administering GABA, or a GABA receptor agonist, to a subject so identified. The invention furthermore relates to methods for assessing a subject's susceptibility to treatment with gamma-aminobutyric acid (GABA) or a GABA receptor agonist, as well as biomarkers to be used in the assessment of the response of a GABA treatment.

### TECHNICAL FIELD

The present invention relates methods assessing a subject's susceptibility to treatment with gamma-aminobutyric acid (GABA) or a GABA receptor agonist, methods of modulating an immune response and use of biomarkers for determining susceptibility for treatment

### INTRODUCTION

Autoimmune diseases are characterized by the subtle defects in the immune system that result in the failure to distinguish between “local” and “foreign” antigens. In such case immune system is set now to attack and destroy the molecules considered as harmful to the organism. These events underlie the pathophysiological mechanisms of the development of many autoimmune syndromes, as diverse as rheumatoid arthritis (RA), type 1 diabetes, multiple sclerosis, etc.

Therefore, the common treatment strategy for autoimmune diseases is a general immunosuppression that would decrease the immune response. Thus, the standard medication scheme applied for the treatment of one of the prototypic syndromes, RA comprises of the first line medicines, such as disease-modifying antirheumatic drugs (DMARD) alone or in combination with glucocorticoids and biologics that target parts of the immune system triggering joint and tissue-damaging inflammation. However, not very high efficacy of these drugs and often side effects necessitates the development of a new generation of efficient and harmless medicines.

Recent developments in this field lead to the discovery that many immune cells, including T-cells, express receptors for neuroactive molecules. Such are, in particular, GABA-R, receptors recognizing y-aminobutyric acid (GABA). GABA is a major inhibitory neurotransmitter that is synthesized from glutamic acid by the glutamate decarboxylase in the brain. In the brain, GABA is made in neurons from the amino acid glutamate by the enzyme glutamic acid decarboxylase (GAD) that is present in two isoforms, GAD65 and 67 (Bu et al., 1992). However, discernible amounts of GABA were also found in the pancreatic islets, the gastrointestinal tract, immune cells. GAD is also found in the insulin-secreting β cells in the pancreatic islets, where GAD65 is one of the main autoantigen in T1D in humans (Kanaani et al., 2015, Bu et al., 1992, Baekkeskov et al., 1990). Interestingly, some immune cells may also produce and release GABA (Fuks et al., 2012, Bhat et al., 2010). Where GABA in blood comes from is still being explored, but the recently discovered drainage system of the brain, the glymphatic system (Plog and Nedergaard, 2017), identifies the brain, in addition to peripheral organs, as a potential source for GABA in blood. In the pancreatic islets, GABA is an auto- and paracrine signaling molecule activating GABA receptors on the endocrine cells and, perhaps, also on immune cells that may enter the islets (Birnir and Korpi, 2007, Kanaani et al., 2015, Caicedo, 2013, Bhandage et al., 2015). Similarly, in blood, immune cells may be regulated by GABA (Bhandage et al., 2015, Bjurstom et al., 2008, Tian et al., 2004, Tian et al., 1999). In T1D, the β cell mass declines and, thereby, also the local source for GABA in the pancreatic islets (Fiorina, 2013, Tian et al., 2013).

GABA activates two types of receptors in the plasma membrane of cells; the GABA_{A} receptors, that are Cl⁻ ion channels opened by GABA, and the G-protein-coupled GABA_{B} receptor (Marshall et al., 1999, Olsen and Sieghart, 2008, Olsen and Sieghart, 2009). The GABA_{A} receptors are pentameric, homo- or heteromeric, receptors formed from 19 known subunit isoforms (α1-6, β1-3, γ1-3, δ, εθ, π, ρ1-3) (Olsen and Sieghart, 2009). In contrast, the GABA_{B} receptor is normally formed as a dimer of the two isoforms identified to date (Marshall et al., 1999, Gassmann et al., 2004). GABA receptors are expressed in immune cells, but their ability to influence the functional phenotype, i.e. proliferation, migration or cytokine secretion, of the cells is still relatively unexplored (Barragan et al., 2015, Jin et al., 2011b).

It has been reported that peripheral administration of GABA or its agonists can modulate the immune response by, for instance, inhibiting antibody production or alter macrophage phagocytosis. Recent reports demonstrated that treatment with GABA can inhibit the development of type 1 diabetes (T1D) in nonobese diabetic mice and treatment with a GABA_{A}-R ligand mitigated experimental autoimmune encephalitis. Moreover, oral GABA administration inhibited the development of disease in the collagen-induced arthritis mouse model of RA. Thus, GABA was found to downregulate both T-cell autoimmunity and APC activity. These results suggest that activation of peripheral GABA-Rs may represent a novel treatment strategy aiming at modulation of T, B cell and APC activities that would be instrumental in amelioration of RA and other inflammatory diseases.

### SUMMARY OF THE INVENTION

The present invention is defined by the appended claims.

In a first aspect, the invention relates to a method for identifying subjects at risk of developing an autoimmune or inflammatory disorder, comprising isolating Peripheral Blood Mononuclear Cells (PBMCs) from a blood sample obtained from said subject;
culturing a subset of said PBMCs in the presence of GABA, or a GABA receptor agonist;
culturing a subset of said PBMCs in the absence of GABA, or a GABA receptor agonist;
measuring the proliferation of said PBMCs in the presence and absence of GABA or GABA receptor agonist;

wherein a reduced proliferation in the presence of GABA or GABA receptor agonist relative the proliferation in the absence of GABA or GABA receptor agonist is indicative of the subject being at risk of developing an autoimmune or inflammatory disorder.

According to a further aspect, a method for identifying subjects at risk of developing an autoimmune or inflammatory disorder, comprising isolating Peripheral Blood Mononuclear Cells (PBMCs) from a blood sample obtained from said subject;
culturing a subset of said PBMCs in the presence of GABA, or a GABA receptor agonist;
culturing a subset of said PBMCs in the absence of GABA, or a GABA receptor agonist;
obtaining a cytokine profile of said PBMCs in the presence and absence of GABA or GABA receptor agonist;

wherein a change in the cytokine profile in the presence of GABA or GABA receptor agonist relative the cytokine profile in the absence of GABA or GABA receptor agonist is indicative of the subject being at risk of developing an autoimmune or inflammatory disorder.

In yet a further aspect, the invention relates to a method of prevention of development of an autoimmune or inflammatory disorder, comprising administering GABA, or a GABA receptor agonist, to a subject identified to be at risk according to the above.

In a further aspect, the present invention relates to a method for assessing a subject's susceptibility to treatment with gamma-aminobutyric acid (GABA) or a GABA receptor agonist, comprising isolating Peripheral Blood Mononuclear Cells (PBMCs) from a blood sample obtained from said subject;
culturing a subset of said PBMCs in the presence of GABA, or a GABA receptor agonist;
culturing a subset of said PBMCs in the absence of GABA, or a GABA receptor agonist;
measuring the proliferation of said PBMCs in the presence and absence of GABA or GABA receptor agonist;

wherein a reduced proliferation in the presence of GABA or GABA receptor agonist relative the proliferation in the absence of GABA or GABA receptor agonist is indicative of the subject being susceptible to treatment with GABA or a GABA receptor agonist.

According to a second aspect, the prevention relates to method for assessing a subject's susceptibility to treatment with gamma-aminobutyric acid (GABA) or a GABA receptor agonist, comprising isolating Peripheral Blood Mononuclear Cells (PBMCs) from a blood sample obtained from said subject;
culturing a subset of said PBMCs in the presence of GABA, or a GABA receptor agonist;
culturing a subset of said PBMCs in the absence of GABA, or a GABA receptor agonist;
obtaining a cytokine profile of said PBMCs in the presence and absence of GABA or GABA receptor agonist;

wherein a change in the cytokine profile in the presence of GABA or GABA receptor agonist relative the cytokine profile in the absence of GABA or GABA receptor agonist is indicative of the subject being susceptible to treatment with GABA or a GABA receptor agonist.

In a further aspect, the invention relates to a method for treatment comprising assessing a subject's susceptibility to treatment with gamma-aminobutyric acid (GABA) or a GABA receptor agonist, comprising performing the method according to the above, and administering GABA or a GABA receptor agonist to said subject only if the subject is indicated as susceptible to treatment with GABA or a GABA receptor agonist.

In yet a further aspect, the invention relates to a method for assessing a subject's responsiveness to treatment with gamma-aminobutyric acid (GABA) or a GABA receptor agonist, comprising measuring the expression of MSMO1, whereby an increased expression of MSMO1 indicates that the subject is responding to the treatment with gamma-aminobutyric acid (GABA) or a GABA receptor agonist.

### DESCRIPTION OF THE FIGURES

FIG. 1: Study of proliferation of the cells when stimulated with anti-CD3 antibodies in the presence and absence of GABA, as well as in the presence of both GABA and Picrotoxin. (A) CD4-positive cells from healthy donors; (B) CD4-positive cells from 10 healthy donors; (C) PBMCs of healthy donors; (D) PBMC from T1D donors.

FIG. 2: Cytokines in plasma from ND and T1D individuals and identification of those that correlate with plasma GABA concentration. (a) Screening of 92 inflammatory cytokines in plasma samples from ND (n=30) and T1D individuals (n=64) by Olink Multiplex PEA inflammation panel I detects expression of 73 cytokines. Data is presented by 2^{NPX} (Normalized Protein Expression) values as floating bars (minimum to maximum) arranged in descending order of mean expression level of cytokines. (b) Inflammatory cytokines with significant change in the expression levels in the plasma of T1D as compared to ND individuals. Data is shown as box and whiskers overlapped with scatter dot plot. (c) Quantification of GABA levels in plasma samples from ND and T1D individuals. (d) Correlation between GABA levels and cytokine levels in plasma samples from ND individuals and T1D individuals. Only cytokines with significant correlation are shown. R values are given in Table S3. *p<0.05, **p<0. 01, ***p<0.001.

FIG. 3: GABA activation of GABAA receptors inhibits proliferation of PBMCs and responder CD4+ T cells from T1D and ND individuals. Effects of GABA and GABAA receptors antagonist, picrotoxin, on proliferation of PBMCs from (a) ND individuals and (b) T1D individuals. (c) Effect of GABA on proliferation of CD4+ T cells from ND individuals identifying GABA non-responder (orange) and GABA responder (magenta) populations of CD4+ T cells. (d) GABA dose-dependent inhibition of proliferation of responder CD4+ T cells. Effects of GABA_{A} (picrotoxin, muscimol, TACA) and GABA_{B} (CGP 52432, baclofen) receptor antagonists (e) and agonists (f) on proliferation of responder CD4+ T cells. (g) Expression of GABA_{A}Rs and GABA_{B}R subunits and (h) chloride transporters in PBMCs from ND individuals and T1D individuals. Data are presented as normalized mRNA expression (2^{-ΔCT}). (i) Single-channel current measurements from CD4+ T cells from ND individuals. In the whole-cell and cell-attached configurations single-channel currents were activated by 500 and 100 nM GABA application, respectively, and inhibited by picrotoxin (PTX, 100 μM). # mark single-channel events with typical current amplitudes at the given Vp. (j) A volcano plot for T cell mRNA sequencing expression data as log2 (fold change in T1D T cells compared to ND T cells) against—log10 (false discovery rate). The p values are shown in Table S5. *p<0.05, **p<0. 01, ***p<0.001.

FIG. 4: Identification of cytokines released into the culture media and effects of GABA treatment on PBMCs cytokine secretion. (a) Screening of 92 cytokines in PBMC media from ND individuals and from T1D individuals by Olink Multiplex PEA inflammation panel I revealed expression of 63 cytokines. No GABA was added to the media. Data are represented by 2^{NPX} values as floating bars (minimum to maximum) arranged in descending order of mean expression level of cytokines. Insert show cytokines with significant change in the expression levels in the media of PBMCs from ND individuals compared with T1D individuals. Data are plotted as a bar graph with mean±SEM. (b-c) Expression of cytokines that are significantly affected by GABA 100 nM treatment of PBMCs from ND individuals (b) and from T1D individuals (c). Data are represented by 2^{NPX} values normalized to controls as a bar graph with mean±SEM. Mean values with SEM and p values are shown in Tables S6 and S7. (d) Classification based on the cellular functions of cytokines that were significantly altered by GABA 100 nM treatment of PBMCs from ND individuals (16 cytokines) and from T1D individuals (49 cytokines).

FIG. 5: Identification of cytokines released into the culture media and effects of GABA treatment on CD4+ T cells cytokine secretion (a) Screening of 92 cytokines in culture media from GABA non-responder and from GABA responder CD4+ T cells by Olink Multiplex PEA inflammation panel I revealed expression of 64 cytokines. No GABA was added to the media. Data are represented by 2^{NPX} values as floating bars (minimum to maximum) arranged in descending order of mean expression level of cytokines. Insert show cytokines with significant change in the expression levels in the media of GABA non-responder and GABA responder CD4⁺ T cells. Data are plotted as a bar graph with mean±SEM. (b-c) Cytokines with significant change in the expression levels after 100 nM GABA (b) or 500 nM GABA(c) treatment of non-responder and responder CD4⁺ T cells, and then in the presence of GABA plus 100 μM picrotoxin (lower panel b, c). Mean and SEM for CXCL11, CCL19 and CCL20 are 1.55±0.51, 1.72±0.74, 1.43±0.51, respectively. Data are represented by 2^{NPX} values normalized to controls as a bar graph with mean±SEM. Mean values with SEM and p values are shown in Tables S8, S9. (d) Classification based on the cellular functions of cytokines that were significantly altered by GABA 100 nM (27 cytokines) and GABA 500 nM (25 cytokines) treatment of responder CD4+ T cells from ND individuals based on their cellular functions.

FIG. 6: GABA and T1D regulate secretion of cytokines. (a) Upper left circle: responder CD4+ T cell cytokines regulated by GABA (100 nM, 500 nM). Upper right circle: T1D PBMC cytokines regulated by GABA (100 nM). Lower circle: cytokines significantly altered in plasma from T1D subjects as compared to ND individuals. (b) The cytokines regulated by 100 nM or 500 nM GABA or both concentrations.

FIG. 7: Standard curve for GABA measured by ELISA. Circles represent the optical density values measured at 450 nM for standard GABA solutions ranging from a concentration 75 nM to 7500 nM provided with kit, n=3. Data is presented as mean±SEM. Squares represent the optical density values provided in quality control report of the kit.

FIG. 8: Protein expression of chloride transporter NKCC1 and GABA_{A}R p2 subunit in PBMCs. Chloride transporter NKCC1 and GABA_{A}R p2 subunit protein bands were detected in PBMC protein extracts from ND (n=3) and T1D (n=4) individuals. GAPDH (Glyceraldehyde 3-phosphate dehydrogenase) served as the loading control and 60 μg proteins were loaded in each lane.

FIG. 9: Expression of GABA_{A} receptors accessory proteins and the insulin receptor in PBMCs from ND and T1D individuals. Data is presented as normalized mRNA expression (2^{-ΔCt}) by box and whiskers with scatter dot plot. *p<0.05, ***p<0.001. GABA-RAP, GABA_{A} receptor-associated protein; GAT3, GABA transporter type 3; BGT1, betaine-GABA transporter; GABA-T, GABA transaminase.

FIG. 10: Correlation of MSMO1 and CYP51A1 expression levels (RNAseq) with plasma GABA concentration, BMI, fasting glucose and HbA1c levels in ND and T1D individuals. The expression of MSMO1 was negatively correlated with GABA concentration, BMI, fasting glucose and HbA1c levels, respectively. The expression of CYP51A1 was also negatively correlated with GABA concentration. *p<0.05, **p<0.01.

FIG. 11: Identification of cytokines released into the culture media by resting and stimulated CD4+ T cells from ND individuals. Screening of 92 cytokines in CD4+ T cells by Olink Multiplex PEA inflammation panel I shows identification of 64 cytokines released by stimulated CD4+ T cells compared with 39 cytokines released by resting CD4+ T cells. Data is represented by 2^{NPX} values as floating bars (minimum to maximum) arranged in descending order of mean expression level of cytokines.

### DEFINITIONS

The term “GABA receptor agonist” refers generally, as used herein, to a compound that directly enhaces the activity of a GABA receptor relative to the activity of the GABA receptor in the absence of the compound. “GABA receptor agonists” useful in the invention described herein include compounds such as GABA, baclofen, muscimol, thiomuscimol, cis-aminocrotonic acid (CACA), bicuculline, CGP 64213, and 1,2,5,6-tetrahydropyridine-4-yl methyl phosphinic acid (TPMPA), homotaurine, bamaluzole, gabamide, GABOB, gaboxadol, ibotenic acid, isoguvacine, isonipecotic acid, phenibut, picamilon, progabide, quisqualamine, progabide acid (SL 75102), pregabalin, vigabatrin, 6-aminonicotinic acid, XP13512 ((±)-1-([(α-isobutanoyloxyethoxy) carbonyl]aminomethyl)-1-cyclohexane acetic acid).

The term “PAM” or “Positive Allosteric Modulator” refers to Positive allosteric modulators (PAMs) of GABAA and are well known to those of skill in the art. Illustrative PAMS include, but are not limited to alcohols {e.g., ethanol, isopropanol), avermectins {e.g., ivermectin), barbiturates {e.g., phenobarbital), benzodiazepines, bromides {e.g., potassium bromide, carbamates {e.g., meprobamate, carisoprodol), chloralose, chlormezanone, clomethiazole, dihydroergolines {e.g., ergoloid (dihydroergotoxine)), etazepine, etifoxine, imidazoles {e.g., etomidate), kavalactones (found in kava), loreclezole, neuroactive steroids {e.g., allopregnanolone, ganaxolone), nonbenzodiazepines (e.g., zaleplon, Zolpidem, zopiclone, eszopiclone), petri chloral, phenols (e.g., propofol), piped dinediones (e.g., glutethimide, methyprylon), propanidid, pyrazolopyridines (e.g., etazolate), quinazolinones (e.g., methaqualone), skullcap constituents (e.g. constituents of Scutellaria sp. including, but not limited to flavonoids such as baicalein), stiripentol, sulfonylalkanes (e.g., sulfonmethane, tetronal, trional), valerian constituents (e.g., valeric acid, valerenic acid), and certain volatiles/gases (e.g., chloral hydrate, chloroform, diethyl ether, sevoflurane). The PAMs used in combination with the GABA receptor activating ligands may exclude alcohols, and/or kavalactones, and/or skullcap or skullcap constituents, and/or valerian or valerian constituents, and/or volatile gases. The PAM may comprise an agent selected from the group consisting of a barbituate, a benzodiazepine, a quinazolinone, and a neurosteroid. Illustrative barbituates include, but are not limited to allobarbital (5,5-diallylbarbiturate), amobarbital (5-ethyl-5-isopentyl-barbiturate), aprobarbital (5-allyl-5-isopropyl-barbiturate), alphenal (5-allyl-5-phenyl-barbiturate), barbital (5,5-diethylbarbiturate), brallobarbital (5-allyl-5-(2-bromo-allyl)-barbiturate), pentobarbital (5-ethyl-5-(1-methylbutyl)-barbiturate), phenobarbital (5-ethyl-5-phenylbarbiturate), secobarbital (5-[(2R)-pentan-2-yl]-5-prop-2-enyl-barbiturate), and the like. Illustrative benzodiazepines include, but are not limited to alprazolam, bromazepam, chlordiazepoxide, clonazepam, clorazepate, diazepam, estazolam, flurazepam, halazepam, ketazolam, lorazepam, nitrazepam, oxazepam, prazepam, quazepam, temazepam, triazolam, and the like. Illustrative neurosteroids include, but are not limited to allopregnanolone, and pregnanolone. Furthermore, the 2-cyano-3-cyclopropyl-3-hydroxy-n-aryl-thioacrylamide derivatives of the group 2-cyano-3-cyclopropyl-3-hydroxy-N-(3-methyl-4-trifluormethyl-phenyl)-thioacrylamide, 2-cyano-3-cyclopropyl-N-(4-fluoro-3-methyl-phenyl)-3-hydroxy-thioacrylamide, 2-cyano-3-cyclopropyl-3-hydroxy-N-(3-methyl-4-nitro-phenyl)-thioacrylamide, 2-cyano-N-(4-cyano-3-methyl-phenyl)-3-cyclopropyl-3-hydroxy-thioacrylamide, 2-cyano-3-cyclopropyl-3-hydroxy-N-(4-trifluoromethanesulfinyl-3-methyl-phenyl)-thioacrylamide, 2-cyano-3-cyclopropyl-3-hydroxy-N-(4-trifluoromethanesulfonyl-3-methyl-phenyl)-thioacrylamide, 2-cyano-3-cyclopropyl-3-hydroxy-N-(3-methyl-4-((trifluoromethyl)thio)phenyl)-thioacrylamide, and 2-cyano-3-cyclopropyl-N-(4-chloro-3-methyl-phenyl)-3-hydroxy-thioacrylamide, disclosed in WO2015140081 may be of use as PAMs in the present invention.

Within the present disclosure, an autoimmune or inflammatory disease may be one chosen from the group comprising of Type 1 Diabetes, presymptomatic Type 1 diabetes of stage 1, presymptomatic Type 1 diabetes of stage 2 allergy, Grave's disease, Hashimoto's thyroiditis, hypoglyceimia, multiple sclerosis, mixed essential cryoglobulinemia, systemic lupus erthematosus, Rheumatoid Arthritis (RA), Coeliac disease, or any combination thereof.

The term “Th1-type of response” refers to an immune reaction leading to the production of cytokines mediating pro-inflammatory functions critical for the development of cell-mediated immune responses. The result is accumulation of blood in dilated, leaky vessels, easing diapedesis of leukocytes into areas of danger and allowing recruitment of innate immune cells and opsonins into the interstitium. Thus Th1 cells cause rubor (redness), tumor (swelling), dolor (pain), and calor (warmth), the 4 cardinal signs of inflammation.

The term “Th2-type of response” refers to an immune reaction leading to the production of cytokines that enhance humoral immunity. Th 2-mediated inflammation is characterized by eosinophilic and basophilic tissue infiltration, as well as extensive mast cell degranulation, a process dependent on cross-linking of surface-bound IgE.

The term “T-regulatory response” refers to activation of regulatory T cells, leading to a suppression of immune responses of other cells, and thus maintaining tolerance to self-antigens.

### DETAILED DESCRIPTION OF THE INVENTION

The results from Examples in the present disclosure identify GABA as a potent regulator of cytokine secretion from human PBMCs and CD4⁺ T cells. GABA altered proliferation and cytokine secretion in a concentration-dependent manner and decreased the release of most of the cytokines. Immunomodulatory submicromolar GABA concentrations are normally present in plasma of both non-diabetic (ND) individuals and Type 1 Diabetes (T1D) subjects.

The present inventors have found that PBMCs from most, but not all, healthy donors do not proliferate differently when cultivated in the presence or absence of GABA, while PBMCs from all donors with Type 1 Diabetes proliferated less in the presence of GABA.

In pancreatic islets where the β cells are intact and secrete GABA, as in ND individuals, the islet interstitial GABA concentrations can be expected to fall within the GABA immunomodulatory range. In contrast, GABA immunosuppression in pancreatic islets of T1D subjects is likely to decrease as the disease progresses and the β cells disappear.

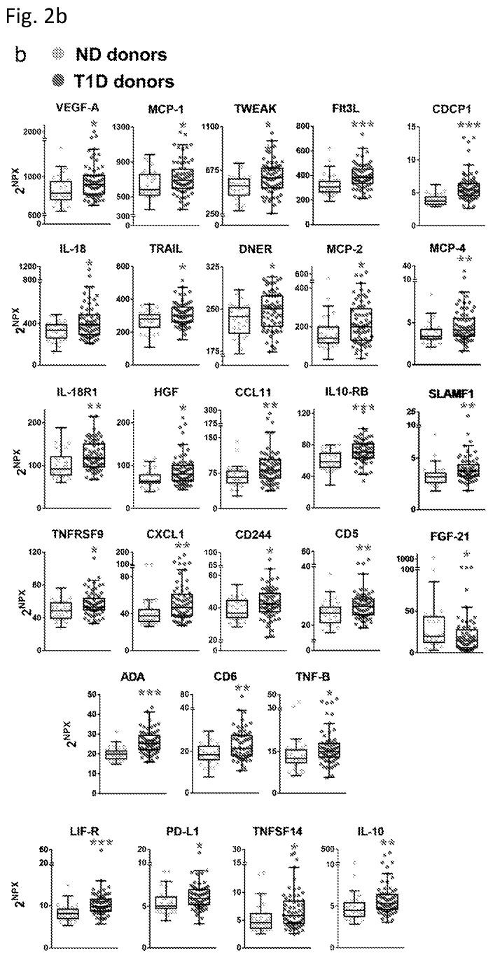

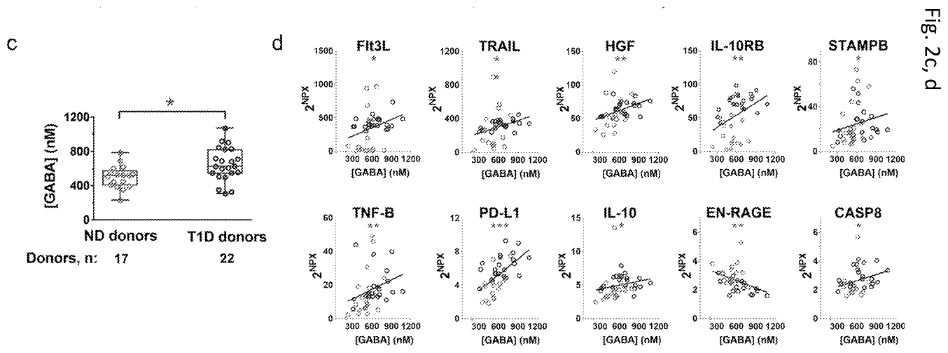

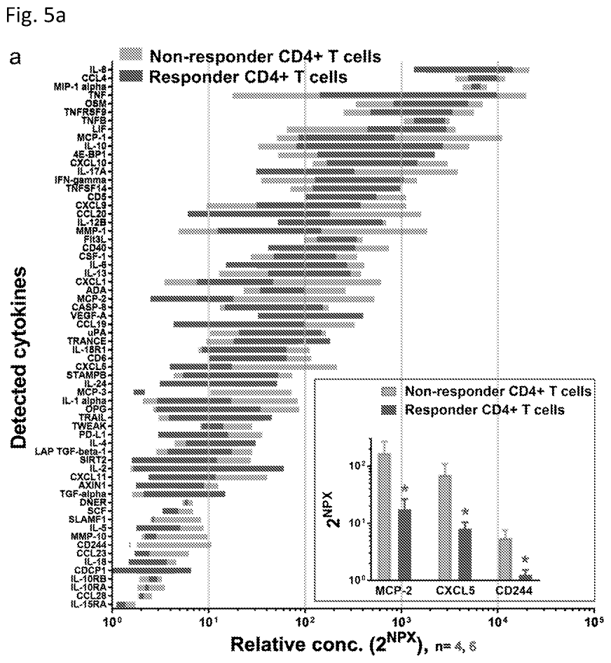

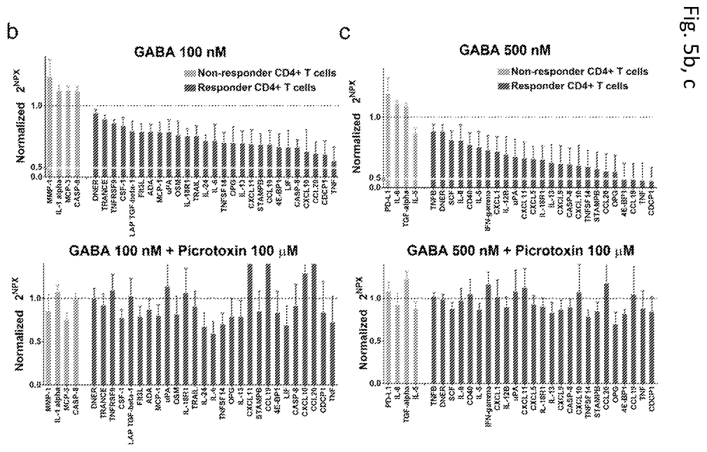

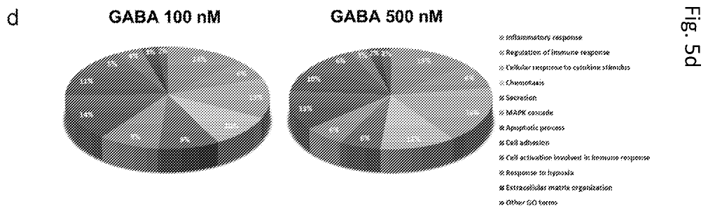

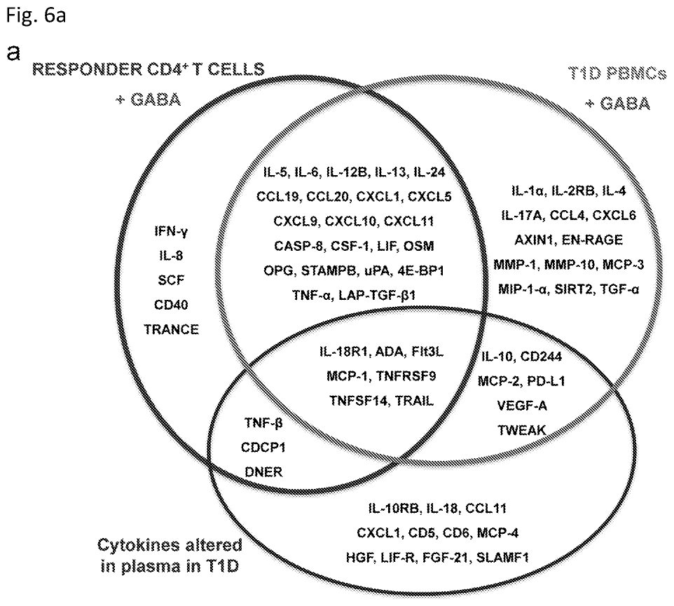

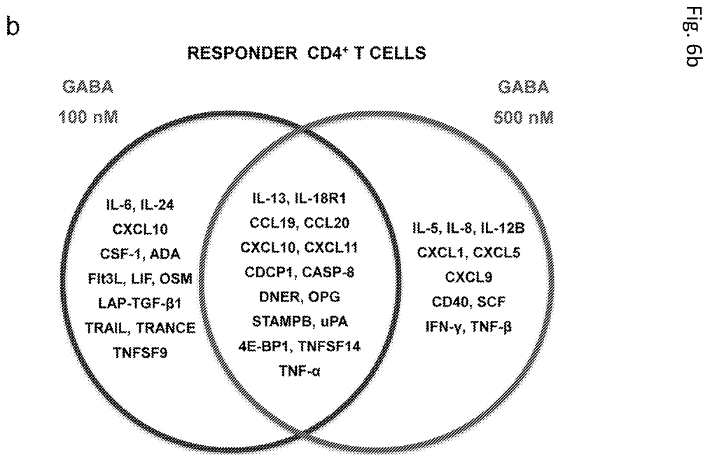

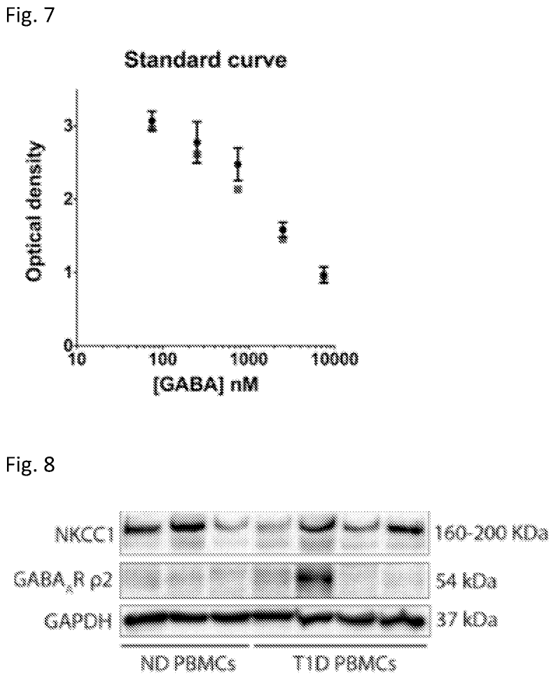

The results presented herein reveal that GABA regulates secretion of a far greater number of cytokines than was previously known. In plasma of T1D subjects, 26 cytokines were increased and of those, 16 were inhibited by GABA in the cell assays (FIGS. 2 and 6). Moreover, of the 10 cytokines in plasma that correlated with the GABA plasma concentration, 7 cytokines were significantly increased in T1D subjects and 5 of those (Flt3L, TRAIL, TNF-β, PD-L1, IL-10) were inhibited by GABA in the cell assays (FIGS. 2d and 6). The few studies that previously have examined effects of GABA on cytokine secretion in immune cells, have only identified a limited number of cytokines e.g. IL-1b (Bhat et al., 2010), IL-2 (Tian et al., 1999), IFNγ (Tian et al., 2004), TNF-α (Duthey et al., 2010) and IL-6, IL-12 (Reyes-Garcia et al., 2007).

The present study reveals that about three times more cytokines were inhibited by GABA in stimulated PBMCs from T1D individuals (47 cytokines) as compared to stimulated PBMCs from ND individuals (16 cytokines). We and others have previously shown that GABA can regulate proliferation of immune cells (Tian et al., 1999, Bjurstom et al., 2008, Jin et al., 2011b, Dionisio et al., 2011, Mendu et al., 2011, Tian et al., 2004). In this study, we used this effect of GABA to divide the stimulated CD4⁺ T cell samples from ND donors into non-responder and responder groups in terms of proliferation and then, examined how these groups differed in cytokine secretion. GABA effectively decreased proliferation and secretion of cytokines only in the responder group. Here GABA decreased secretion of 37 cytokines in a concentration-dependent manner. Of the inhibited cytokines from T1D stimulated PBMCs and the responder cells, 29 cytokines were common to both cell populations (FIG. 6a). Although the GABA-induced immunosuppression was somewhat similar for the responder and T1D cell populations, it clearly differed for a number of specific cytokines (FIG. 6a). Furthermore, GABA did not decrease proliferation of stimulated ND PBMCs nor proliferation or cytokine secretion in the non-responder T cell population. The dramatic effect GABA had on stimulated T1D PBMCs and responder T cell but not on non-responder T cells, implies that GABA is a central molecule determining cytokine secretion in primed, activated cells. It is possible that GABA has a homeostatic role in the immune system acting as a “brake” but not a “full stop”.

According to the invention, a responder in the non-diabetic donor group, as discussed herein, is identified as a subject at at risk of developing an autoimmune or inflammatory disorder.

Thus, the invention provides for a method for identifying a subject at risk of developing an autoimmune or inflammatory disorder, comprising isolating Peripheral Blood Mononuclear Cells (PBMCs) from a blood sample obtained from said subject; culturing a subset of said PBMCs in the presence of GABA, or a GABA receptor agonist; culturing a subset of said PBMCs in the absence of GABA, or a GABA receptor agonist; and measuring the proliferation of said PBMCs in the presence and absence of GABA or GABA receptor agonist; wherein a reduced proliferation in the presence of GABA or GABA receptor agonist relative the proliferation in the absence of GABA or GABA receptor agonist is indicative of the subject being at risk of developing an autoimmune or inflammatory disorder.

Yet further, the invention provides for a method for identifying subjects at risk of developing an autoimmune or inflammatory disorder, comprising isolating Peripheral Blood Mononuclear Cells (PBMCs) from a blood sample obtained from said subject; culturing a subset of said PBMCs in the presence of GABA, or a GABA receptor agonist; culturing a subset of said PBMCs in the absence of GABA, or a GABA receptor agonist; obtaining a cytokine profile of said PBMCs in the presence and absence of GABA or GABA receptor agonist; wherein a change in the cytokine profile in the presence of GABA or GABA receptor agonist relative the cytokine profile in the absence of GABA or GABA receptor agonist is indicative of the subject being at risk of developing an autoimmune or inflammatory disorder.

According to one embodiment, the expression of CDCP1 and TNF is studied to determine if the subject is a GABA responder. According to this embodiment, a significant decrease (p<0.05) of the expression of CDCP1 and TNF in the presence of GABA or GABA receptor agonist relative the expression in the absence of GABA or GABA receptor agonist, is indicative of the subject being at risk of developing an autoimmune or inflammatory disorder.

The present invention also relates to a method of prevention of development of an autoimmune or inflammatory disorder, comprising administering GABA, or a GABA receptor agonist, to a patient subject identified to be at risk of developing said autoimmune or inflammatory disorder, according to the above.

This invention furthermore provides a method for treating a human subject afflicted with an autoimmune or inflammatory disease with a pharmaceutical composition comprising GABA, comprising the steps of determining whether the human subject is a GABA responder by evaluating a biomarker based on the ability of GABA to inhibit T cell proliferation, in the blood of the human subject and administering the pharmaceutical composition comprising GABA to the human subject only if the human subject is identified as a GABA responder.

According to one embodiment of the invention, a statistically significant reduction of proliferation in the presence of GABA or GABA receptor agonist relative the proliferation in the absence of GABA or GABA receptor agonist, such as a reduction by 10, 20, 30, 40, 50, 60, 70, 80, or 90%, is indicative of the subject being susceptible to treatment with GABA.

The invention further provides a method for treating a human subject afflicted with an autoimmune or inflammatory disease with a pharmaceutical composition comprising GABA, comprising the steps of determining whether the human subject is a GABA responder by evaluating a biomarker based on the ability of GABA to change the cytokine expression profile, in the blood of the human subject and administering the pharmaceutical composition comprising GABA to the human subject only if the human subject is identified as a GABA responder by such a changed cytokine expression profile.

As can be seen in the results under Experiment 2 below, there are many cytokine expression levels that are changed in response to a GABA treatment in the GABA responders. Thus, any of the cytokines indicated to have an altered expression level could be used to determine if the subject is a GABA responder. According to one embodiment, the expression of CDCP1 and TNF is studied to determine if the subject is a GABA responder. According to this embodiment, a significant decrease (p<0.05) of the expression of CDCP1 and TNF in the presence of GABA or GABA receptor agonist relative the expression in the absence of GABA or GABA receptor agonist, is indicative of the subject being susceptible to treatment with GABA.

This invention also provides a method of predicting clinical responsiveness to GABA therapy in a human subject afflicted with an autoimmune or inflammatory disease, the method comprising evaluating a biomarker based on the ability of GABA to inhibit T cell proliferation or to change the cytokine expression profile, in the blood of the human subject, to thereby predict clinical responsiveness to GABA.

GABA inhibited cytokines involved in chemotaxis in stimulated T1D PBMCs more than in ND PBMCs cells. When the GABA concentration was increased from 100 to 500 nM for the stimulated responder T cells, the prominence of inhibited cytokines associated with secretion and MAPK was decreased. In contrast, inhibition of cytokines that affect either the cellular response to cytokine stimulus or regulate the immune response increased in 500 nM GABA. The specific profile of cytokines regulated by GABA indicates that the 100 nM GABA response tended to modulated levels of Th2-type cytokines, whereas the 500 nM GABA inhibited both Th1- and Th2-type cytokine release (FIG. 6a, b). The results are consistent with a concentration-dependent immunomodulatory effects of GABA.

Thus, a human subject may initially be treated with a first dose of GABA or GABA agonist. If the T-cell proliferation is reduced following such a treatment, the human subject is responding to the GABA treatment and the dosage administered may be maintained. However, if the subject does not respond to the above mentioned first dose, the dose may be increased to a second dose GABA or GABA agonist. Thus, the dose may be increased until the desired inhibition of a Th2-type of response is observed in the subject.

For a nondiabetic individual, such a response may be indicative of the subject being at risk of developing an autoimmune or inflammatory disease. The response to the treatment indicates that the subject has GABA reactive T-cells, which are common in subjects with Type 1 Diabetes. Thus, such a result indicates that the individual also have a higher risk of developing an autoimmune or inflammatory disease that is driven by GABA reactive T cells. In such a case, GABA or a GABA agonist may be administrered as a preventive treatment. Alternatively, the presence of such a response may be used in a regularly preformed monitoring of the subject, in order to early detect the onset of such a disease.

In particular, the inventors have shown that treatment with a first dose may inhibit a Th2 type of response by modulating and inhibiting the release of Th2 type cytokines.The inhibition of a Th2 type of response is preferably assessed according to one of the methods of the invention as disclosed above However, treatment with a second, higher dose may inhibit both a Th2 and a Th1 type of response, by modulating and inhibiting the release of both Th2 and Th1 cytokines. The inhibition of both a Th1 and a Th2 type of response is equally assessed according to one of the methods according to the invention as disclosed above.Thus an immuneresponse may be regulated and modulated in a dose dependent manner.

One of the key discoverys being used within the methods of the present invention is that there is a dose dependent response in a subject following treatment with a GABA or GABA agonist, whereby the subjects response may be regulated and modulated. Thereby it is possible to tailor make a treatment for a subject, depending on the response that is desired or required.

This invention also provides a method for treating a human subject afflicted with an autoimmune or inflammatory disease with a pharmaceutical composition comprising GABA, comprising the steps of determining whether the human subject is a GABA responder by evaluating a biomarker based on the ability of GABA to inhibit T cell proliferation, in the blood of the human subject, and continuing administration of the pharmaceutical composition if the human subject is identified as a GABA responder, or modifying the administration of the pharmaceutical composition to the human subject if the human subject is not identified as a GABA responder.

By treating a subject with a GABA or GABA agonist a Th2 type of response may be inhibited. The Examples below support this and it is clear that cytokines connected to to Th2 type of response are downregulated. By increasing the dose of GABA or GABA agonist, not only Th2 but also a Th1 type of response may inhibited. Thus, it is possible to regulate and modulate an immune response in a human subject by regulating the dose of GABA or GABA agonist being used. Thus, According to one embodiment of the methods of the invention, a first dose may be used to to induce a T-regulatory response for the subject. According to a further embodiment, the T-regulatory response may be measured as an increase in IL-4 secretion following GABA treatment. According to another embodiment of the methods of the invention, a second dose, increased in relation to the first dose, may be used to inhibit both a Th2 type and a Th1 type of response in a subject.

It is important not to increase the dose so that an interstitial concentration above 1000 nM is achieved, as this will shut down the GABA receptor and thus the responsiveness for the treatment of the subject.

Thus, in one aspect the invention relates to a method for treatment wherein GABA, and optionally a PAM, is administered in an amount effective to inhibit a Th2-type of response for the subject.

In a further aspect, the invention relates to a method for treatment wherein GABA, and optionally a PAM, is administered in an amount effective induce a T-regulatory response for the subject. The T-regulatory response may be measured as an increase in IL-4 secretion following GABA treatment.

In a further spect, the invention relates to a method for treatment wherein GABA, and optionally a PAM is administered in an amount effective to inhibit a Th2 type and a Th1 type of response for the subject.

This invention also provides a method of predicting clinical responsiveness to GABA therapy in a human subject determined to have a high risk of being diagnosed with an autoimmune or inflammatory disease, the method comprising evaluating a biomarker based on the ability of GABA to inhibit T cell proliferation, in the blood of the human subject, to thereby predict clinical responsiveness to GABA.

The inventors have shown that there is a dose response dependency between the concentration of GABA or GABA agonist used, and the effect achieved on the immune system. Also the subunits that are expressed in immune cells are dependent on the concentration of GABA or GABA agonist administered. Thus the invention also provides for a method of treatment of a human subject afflicted with an autoimmune or inflammatory disease, to modulate the immune response in said subject.

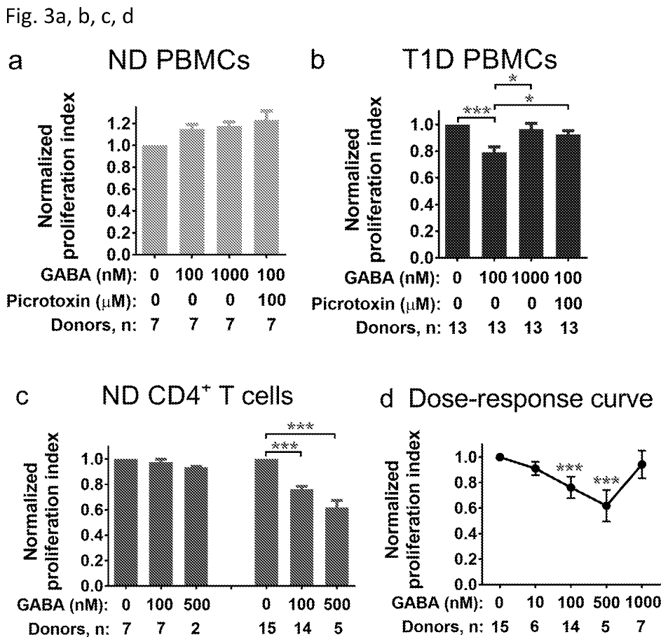

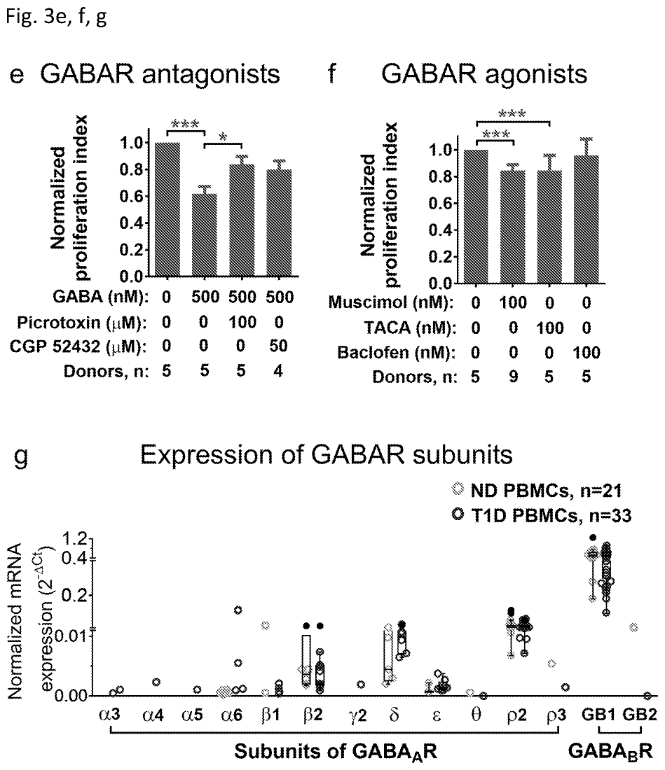

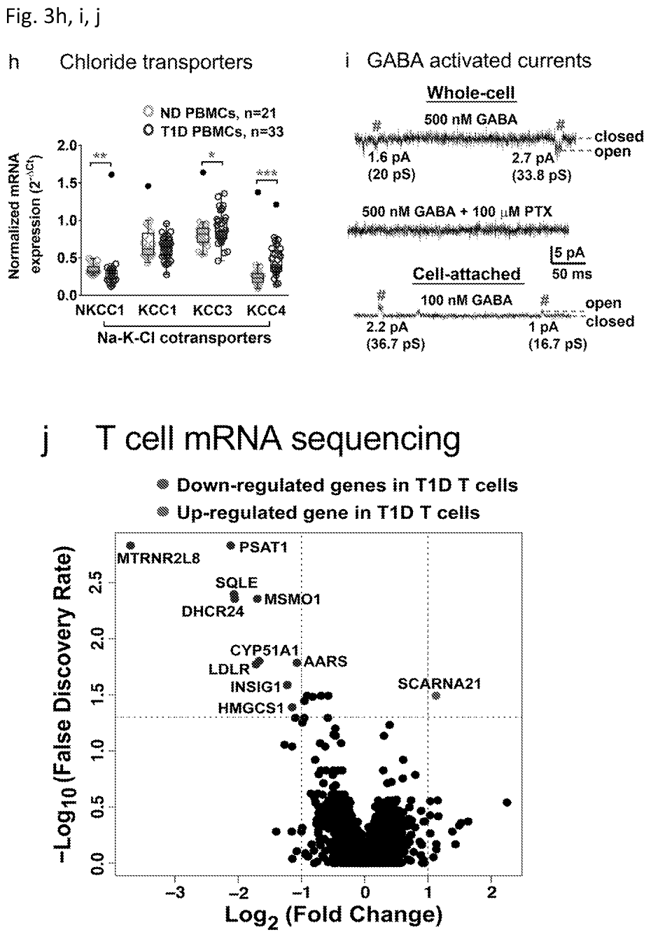

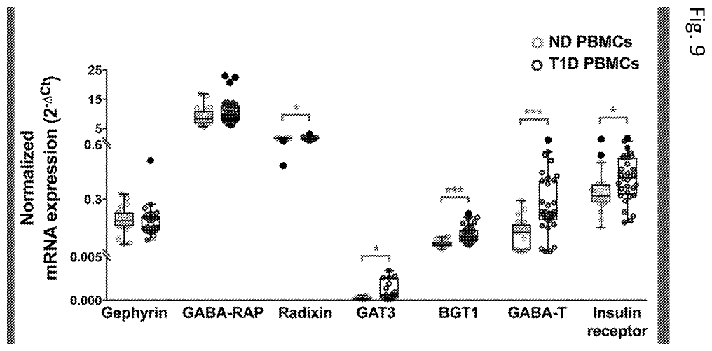

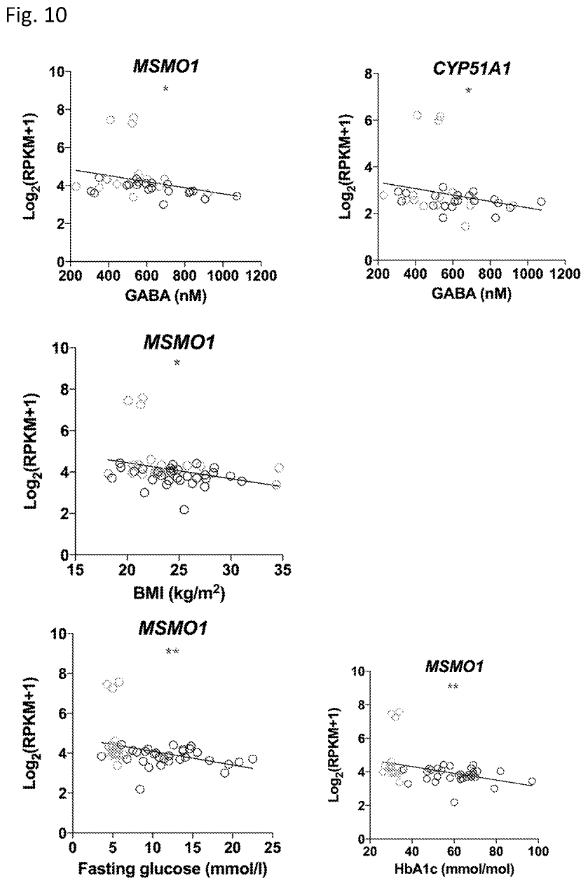

Of the 10 genes down-regulated more than two-fold in the CD3⁺ T cells from T1D individuals, six were associated with cholesterol biosynthesis (FIG. 3j) and included MSMO1 and CYP51A1 that correlated negatively with the plasma GABA concentrations (FIG. 10). MSMO1 further correlated negatively with HbA1c, fasting glucose and BMI raising the question of its potential suitability as a biomarker in T1D. Non-random distribution of proteins in the cell membranes are dependent on e.g. cholesterol and the actin cytoskeleton (Goyette and Gaus, 2017). The T cell receptor resides in lipid rafts that are microdomains within the plasma membrane and where cholesterol is an essential component and contributes to membrane fluidity and signal transduction (Kidani and Bensinger, 2016, Hubler and Kennedy, 2016). The change in cholesterol biosynthesis genes observed in the T1D T cells are likely to have an impact on signaling processes associated with signaling complexes located in the cell membrane.

Thus, the present disclosure also provides for a method for assessing a subject's responsiveness to treatment with gamma-aminobutyric acid (GABA) or a GABA receptor agonist, comprising measuring the expression of MSMO1, whereby an increased expression of MSMO1 indicates that the subject is responding to the treatment with gamma-aminobutyric acid (GABA) or a GABA receptor agonist.

To the inventors knowledge, this is the first time it is shown that GABA may modulate an immuneresponse by modulating the expression of cytokines, both pro- and anti-inflammatory cytokines. Additionally, to the inventors knowledge, this is the first time this regulation of the immune response exerted by GABA has been shown in human cells.

Furthermore, this is the first time that a dose dependent response on GABA has been observed. In particular, it is the first time it has been shown that a dose dependent response in the sense that a Th2 or Th2 and Th1 response, respectively, may be inhibited by increasing or decreasing the dose of GABA administered. Thus it is possible to modulate the immune response of a subject in different direction by regulating the dose of GABA used in the treatment.

### EXPERIMENTAL SECTION

### Example 1

Peripheral Blood Mononuclear cells (PBMCs) are isolated from blood from donors diagnosed with Type-1 diabetes and healthy controls. Alternatively, CD4-positive cells (T-cells) are further isolated from PBMCs by e.g. MACS beads, FACS or similar technology. Cells from each donor are then split and an appropriate number of cells (e.g. 10̂6) are cultured with T-cell stimulating anti-CD3 antibodies both in the presence and absence of 100 nM GABA. A reasonable replication, such as 3 cultures of each condition, is performed. A normalized proliferation value for each culture is then calculated by a standard proliferation measurement method, such as by CFSE staining or radioactive thymidine incorporation (requiring additional factors be added during culture) or by staining for proliferation markers. Proliferation values of cultures with and without GABA are compared. If the proliferation value of cells cultured with GABA is less than 90% of the value of the cells culture without GABA, i.e. GABA has reduced the proliferation by more than 10%, the test is considered to have a positive outcome.

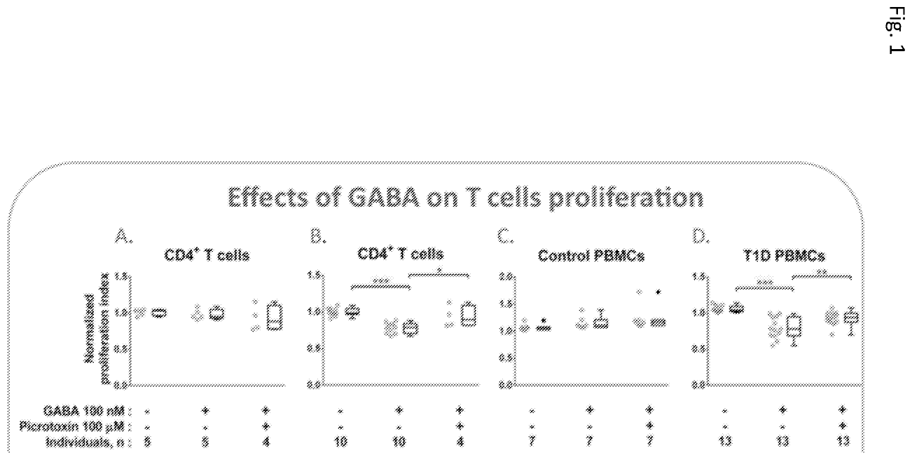

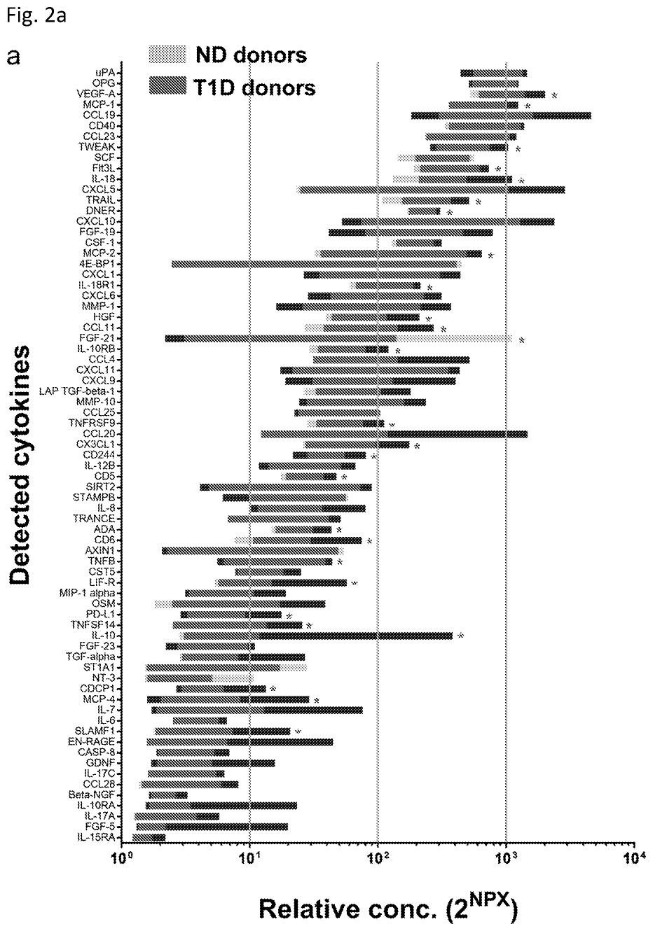

In the FIG. 1, the proliferation of cells retrieved from 13 T1D subjects and 15 healthy controls were inspected. PMBCs were isolated from blood samples and proliferation and the proliferation of the cells when stimulated with anti-CD3 antibodies in the presence and absence of GABA was investigated, as well as in the presence of both GABA and Picrotoxin (a GABA receptor inhibitor). Additionally, CD4-positive cells were isolated from PBMCs of healthy donors and their proliferation when stimulated with anti-CD3 antibodies in the presence and absence of GABA was investigated, as well as in the presence of both GABA and Picrotoxin. A normalized proliferation index was calculated for each culture.

FIG. 1 shows that CD4-positive cells from 5 of the healthy donors did not proliferate less in the presence of GABA (A), while CD4-positive cells from 10 of the healthy donors did proliferate less in the presence of GABA (B), an effect which was reversed by the presence of Picrotoxin. The PBMCs of the 15 healthy donors did not proliferate differently in the presence of GABA (C), but PBMCs from all 13 T1D donors proliferated less in the presence of GABA (D), an effect that was reversed by the presence of Picrotixin.

### Example 2

GABA regulates release of inflammatory cytokines from peripheral blood mononuclear cells and CD4⁺ T cells and is immunosuppressive in type 1 diabetes.

1. Materials and Methods

1.1. Study Individuals and Ethical Permits

The study was approved by Regional Ethical Review Board in Uppsala, and the reported investigations were carried out in accordance with the principles of the Declaration of Helsinki as revised in 2000. The study includes 30 healthy controls and 64 T1D subjects. All participants signed a written consent form before entering the study. The participants were recruited at Uppsala University Hospital. Demographic characteristics of the participants are summarized in Table S1. All the participants were screened for islet autoantibodies (GAD and islet antigen-2, IA2), which were not present in any of the healthy controls. None of the healthy controls had a first degree relative diagnosed with T1D. None of the participants was ill from, or had recently recovered from, an infectious disease. All blood samples were collected in the morning after an overnight fasting under standardized conditions. Routine lab parameters were analyzed at the Central Clinical Chemistry Laboratory, Uppsala University Hospital. The venous blood samples were collected in EDTA tubes and processed for further experimentation.

TABLE S1
Descriptive data of study participants

| | Healthy control | Type 1 Diabetes | |
|---|---|---|---|
| Parameter | (n = 30) | (n = 64) | p-value |
| Age (years) | 25 ± 0.7 | 29 ± 0.8 | 0.005^{a} |
| Age at onset of | NA | 14 ± 1 | NA |
| T1D (years) | | | |
| Disease duration | NA | 15 ± 1 | NA |
| (years) | | | |
| Sex (n, %) | 12 male (40%) | 37 male (58%) | 0.13^{b} |
| BMI (kg/m²) | 23.2 ± 0.69 | 24.8 ± 0.37 | 0.0009^{a} |
| Fasting glucose | 5.4 ± 0.09 | 11.1 ± 0.6 | <0.0001^{c} |
| (mmol/l) | | | |
| HbA1c (mmol/mol, | 31 ± 0.4 | 60 ± 1.5 | <0.0001^{c} |
| NGSP %) | (5 ± 0.04%) | (7.6 ± 0.14%) | |
| Creatinine (μmol/l) | 71.9 ± 1.8 | 68.7 ± 1.4 | 0.2^{c} |
| Glomerular filtration | 105 ± 3.4 | 114 ± 2.4 | 0.02^{a} |
| rate (ml/min) | | | |
| GAD positive (n, %) | 0 | 44 (69%) | <0.0001^{b} |
| IA2 positive (n, %) | 0 | 24 (38%) | <0.0001^{b} |
| GAD and IA2 | 0 | 18 (28%) | 0.0005^{b} |
| positive (n, %) | | | |

Data is presented as means ± SEM.
Glomerular filtration rate was calculated based on the Modification of Diet in Renal Disease (MDRD) formula.
For glutamate acid decarboxylase (GAD) antibodies, 5 IE/mland for islet antigen-2 (IA2) antibodies, 15 kE/I were used as the cut-off value.
Normality distribution of demographic data was tested by the D'Agostino & Pearson omnibus normality test.
For comparison of non-normality distributed data a non-parametric Mann-Whitney test was applied^{a} and a two-tailed Student's T-test was applied for normally distributed data^{c}.
Fisher's exact test was applied for comparison of categorical data^{b}.
All p-values < 0.05 were considered statistically significant.
NA-not applicable.

1.2. Plasma, PBMCs and T Cell Isolation

Plasma, PBMCs and T cells were isolated from freshly derived blood samples and CD4⁺ T cells from buffy coats as previously described (Bhandage et al., 2015, Bhandage et al., 2017). The plasma was isolated by centrifugation at 3,600 rpm for 10 min at 4° C. directly after collection of blood, and immediately frozen at −80° C. The blood samples or buffy coats were diluted in 1:1 ratio in MACS buffer (Miltenyi Biotec, Madrid, Spain), and layered on Ficoll-paque plus (Sigma-Aldrich, Hamburg, Germany). Briefly, the samples were then subjected to density gradient centrifugation at 400 g for 30 min at room temperature. The PBMCs were carefully withdrawn and washed twice in MACS buffer. A portion of PBMCs was saved in RNAlater (Sigma-Aldrich) at −80o C for mRNA extraction for qPCR, and other portions were used for either proliferation experiments or isolation of T cells using human CD3 MicroBeads and human CD4⁺ T Cell Isolation Kits (Miltenyi Biotec). The CD3+ T cells were used for RNA sequencing, and the CD4⁺ T cells were used for proliferation and electrophysiological patch-clamp experiments.

1.3. Total RNA Isolation, Real-Time Quantitative Reverse Transcription PCR and Western Blot Analysis.

Total RNAs were extracted with RNA/DNA/Protein Purification Plus Kit (Norgen Biotek, Ontario, Canada). The real-time qPCR method has been described previously (Schmittgen and Livak, 2008, Bhandage et al., 2015, Kreth et al., 2010, Ledderose et al., 2011, Bhandage et al., 2017. The extracted total RNA was quantified using Nanodrop (Nanodrop Technologies, Thermo Scientific, Inc., Wilmington, Del., USA). Then, 1.5 μg RNA was treated with 0.6 U DNase I (Roche, Basel, Switzerland) for 30 min at 37° C. to degrade genomic DNA in the sample, and then with 8 mM EDTA for 10 min at 75° C. for inactivation of DNase I enzyme. The cDNA was then synthesized using Superscript IV reverse transcriptase (Invitrogen, Stockholm, Sweden) in a 20 μl reaction mixture using standard protocol provided by manufacturer. To confirm efficient degradation of genomic DNA by DNase I treatment, we performed reverse transcriptase negative reaction which did not yield any amplification in real-time PCR, confirming the absence of genomic DNA contamination. The gene-specific primer pairs are listed in Table S2. The real-time qPCR amplification was performed on an ABI PRISM 7900 HT Sequence Detection System (Applied Biosystems) in a standard 10 μl reaction with an initial denaturation step of 5 min at 95° C., followed by 45 cycles of 95° C. for 15 s, 60° C. for 30s and 72° C. for 1 min, followed by melting curve analysis.

TABLE S2
Primers for RT-qPCR

| | | | |
|---|---|---|---|
| | | | Amplicon |
| | Forward | Reverse | Size |
| Genes | Primer Sequence | Primer Sequence | (bp) |

Endogenous control

| | | | |
|---|---|---|---|
| TBP | GAGCTGTGATGTGAAGTTTCC | TCTGGGTTTGATCATTCTGTAG | 177 |
| IPOB | GCAAAGGAAGGGGAATTGAT | CGAAGCTCACTAGTTTTGACCC | 91 |

19 GABA_{A} receptor subunit genes

| | | | |
|---|---|---|---|
| GABRA1 (α1) | GTCACCGTTTCGGACCCG | AACCGGAGGACTGTCATAGGT | 119 |
| GABRA2 (α2) | GTTCAAGCTGAATGCGCAAT | ACCTAGAGCCAATCAGGAGCA | 160 |
| GABRA3 (α3) | CAACTTGTTTCAGTTCATTCATCC | CTTGTTTGTGTGATTATCATCT | 102 |
| | TT | TCTTAGG | |
| GABRA4 (α4) | TTGGGGGTCCTGTTACAGAAG | TCTGCCTGAAGAACACATCCA | 105 |
| GABRA5 (α5) | TTGGATGGCTACGACAACAGA | GTCCTCACCTGAGTGATGCG | 62 |
| GABRA6 (α6) | ACCCACAGTGACAATATCAAAAGC | GGAGTCAGGATGCAAAACAATCT | 67 |
| GABRB1 (β1) | TGCATGTATGATGGATCTTCG | GTGGTATAGCCATAACTTTCGA | 88 |
| GABRB2 (β2) | GCAGAGTGTCAATGACCCTAGT | TGGCAATGTCAATGTTCATCCC | 137 |
| GABRB3 (β3) | CAAGCTGTTGAAAGGCTACGA | ACTTCGGAAACCATGTCGATG | 108 |
| GABRG1 (γ1) | CCTTTTCTTCTGCGGAGTCAA | CATCTGCCTTATCAACACAGTT | 91 |
| | | TCC | |
| GABRG2 (γ2) | CACAGAAAATGACGGTGTGG | TCACCCTCAGGAACTTTTGG | 138 |
| GABRG3 (γ3) | AACCAACCACCACGAAGAAGA | CCTCATGTCCAGGAGGGAAT | 113 |
| GABRD (δ) | CTTTGCTCATTTCAACGCC | TTCCTCACGTCCATCTCTG | 86 |
| GABRE (ε) | ACAGGAGTGAGCAACAAAACTG | TGAAAGGCAACATAGCCAAA | 107 |
| GABRQ ( [symbol] ) | CCAGGGTGACAATTGGCTTAA | CCCGCAGATGTGAGTCGAT | 63 |
| GABRP ( [symbol] ) | CAATTTTGGTGGAGAACCCG | GCTGTCGGAGGTATATGGTG | 110 |
| GABRR1 (ρ1) | AAAGGCAGGCCCCAAAGA | TCAGAATTGGGCTGACTTGCT | 78 |
| GABRR1 (ρ2) | Hs00366687_m1 from | | 94 |
| | Applied Biosytems | | |
| GABRR2 (ρ3) | TACAGCATGAGGATTACGGT | CAAAGAACAGGTGTGGGAG | 81 |
| GABRR3 (ρ4) | TGATGCTTTCATGGGTTTCA | CGCTCACAGCAGTGATGATT | 111 |

2 GABA [symbol] receptor subunit genes

| | | | |
|---|---|---|---|
| GABBR1 (GABA-B1) | TGGCATGGACGCTTATCGA | GATCATCCTTGGTGCTGTCATAGT | 78 |
| GABBR2 (GABA-B2) | GAGTCCACGCCATCTTCAAAAAT | TCAGGATACACAGGTCGATCAGC | 108 |

6 chloride transporter genes

| | | | |
|---|---|---|---|
| SLC12A2 (NKCC1) | TGGGTCAAGCTGGAATAGGTC | ACCAAATTCTGGCCCTAGACTT | 161 |
| SLC12A1 (NKCC2) | TCAGGAGATTTGGAGGATCCC | ACCCCTAAGTAGGCAACAGTG | 86 |
| SLC12A4 (KCC1) | CCTCCCGTGTTTCCGGTATG | CAGGAGTCGGTCGTAAGGTTG | 155 |
| SLC12A6 (KCC2) | GGAAGGAAATGAGACGGTGA | TCCCACTCGTGTCCACAATC | 200 |
| SLC12A8 (KCC3) | GGATGTCATCGAGGACCTGAG | TCGAGCTTTCTTATGTCCGTC | 82 |
| SLC12A7 (KCC4) | ATCTACTTCCCTTCCGTGACC | TCTGTGCATCCTTGAGGTCC | 70 |

GAGA_{A} receptor accessory proteins

| | | | |
|---|---|---|---|
| ABAT (GABA-T) | TGAAATACCCTCTGGAAGAG | CAATCAGATCCTCCACCTC | 80 |
| GABARAP | AGGCTCCCAAAGCTCGGATA | AATTCGCTTCCGGATCAAGA | 100 |
| Gep [symbol] | TGATCCTTACTAACCACGACCA | TTTATCCCACTGCGGTCTTC | 87 |
| Rad [symbol] | TGGAACGTCTAAAACAAATTGAAG | TTTGCTCGTTTTCGTTCTTG | 106 |
| GAT1 | GGGAGCTACAACTCTTTCCACAAC | CGAATTGATGCAGCAGACGAT | 88 |
| GAT2 | TGGCAGCAGCTTCACTAAGGTGG | GTTGTTCCAGTGCCCCCGCT | 135 |
| GAT3 | ATGCCACCTCCCCTGTCAT | TGCTCGATCCCGTCAGAGAT | 67 |
| BGT1 | ACGACCTGCAACAACTTTTGG | GCTCCTGAGTGGTTCAGAAAGTC | 62 |
| GAD65 | CTACGCGTTTCTCCATGCAA | GCCAAAGTGGGCCTTTCTC | 63 |
| GAD67 | GCGGACCCCAATACCACTAAC | CACAAGGCGACTCTTCTCTTC | 144 |

Islet hormone receptor

| | | | |
|---|---|---|---|
| Insulin receptor | CCTTGGAAATTGGGAACTACT | GGTTGTGTTTGCTCCAGTC | 81 |

[symbol]

indicates data missing or illegible when filed

Protein extraction from PBMC samples was performed using RNA/DNA/Protein Purification Plus Kit (Norgen Biotek, Ontario, Canada). Protein amounts were quantified using the RC DCTM protein assay kit (Bio-Rad, USA) in Multiskan MS plate reader (Labsystems, Vantaa, Finland), and the concentration was calculated by plotting standard curve. Protein samples (60 μg) were subjected to SDS-PAGE using 10% polyacrylamide gels and transferred to PVDF membranes (Thermofisher Scientific, Stockholm, Sweden). The membranes were blocked with 5% non-fat milk powder in Tris buffered saline containing 0.1% Tween (TBS-T) for 1 h and incubated overnight at 4° C. with primary antibodies against NKCC1 (1:2000; Cell Signaling Technology, Cat No. 8351), GABAAR p2 (1:500; Abcam, Cat No. ab83223) and GAPDH (1:3000; merckmillipore, Cat No. ABS16). After 3 washings with TBS-T, the membranes were further incubated with horseradish peroxidase-conjugated secondary antibody (1:3000; Cell Signaling Technology, Cat No. 7074) for 2 h and then the immunoreactive protein bands were visualized by enhanced chemiluminescence (ECL) detection kit (Thermofisher Scientific, Stockholm, Sweden).

1.4. Determination of GABA Concentration

Plasma samples were thawed, and the level of GABA was measured using an ELISA kit (LDN Labor Diagnostika Nord, Nordhorn, Germany) as per manufacturer's guidelines (Fuks et al., 2012, Abu Shmais et al., 2012, El-Ansary et al., 2011, Lee et al., 2011). Briefly, the plasma samples and standards provided in the kit were extracted on extraction plate, derivatized using equalizing reagent and subjected standard competitive ELISA in GABA coated microtiter strips. The absorbance of the solution in the wells was read at 450 nm within 10 min using a Multiskan MS plate reader (Labsystems, Vantaa, Finland). We used 620 nm as a reference wavelength. The outcome of the assay, optical density values, were used to plot the standard curve for each run, which were then used to interpolate the GABA concentration of the samples. The readout obtained by the GABA standards in the kit was compared to and agreed with the standards in the quality control (QC) report from the company (FIG. 2).

1.5. Electrophysiology

GABA-activated currents were recorded by the patch-clamp technique as previously described (Bjurstom et al., 2008, Jin et al., 2011a). Extracellular recording solution contained (in mM): 145 NaCl, 3 KCl, 1 CsCl, 1 CaCl₂, 1 MgCl₂, 10 glucose and 10 TES; the pH was adjusted to 7.4 with NaOH. To record in the whole-cell configuration, the pipette solution contained (in mM): 136 CsCl, 20 KCl, 1 MgCl₂, 3 MgATP and 10 TES; pH was adjusted to 7.3 with CsOH. The pipette solution for the cell-attached configuration contained (in mM): 69 NaCl, 5 KCl, 75 CsCl, 1 CaCl₂, 1 MgCl₂ and 10 TES; pH was adjusted to 7.4 with NaOH. Saclofen (a GABAB receptor antagonist, 200 μM) and GABA (100 nM) were used in the experiments. The pipette potential (Vp) was −80 mV (hyperpolarizing) in the whole-cell configuration and −60 mV (depolarizing) in the cell-attached configuration.

1.6. Proliferation Assay

The proliferation of freshly isolated human PBMCs or CD4⁺ T cells was evaluated with MTT (3-(4,5-Dimethylthiazol-2-yl)-2,5-Diphenyltetrazolium Bromide) assay (Ring et al., 2012). Cells were suspended in complete medium (RPMI 1640 supplemented with 2 mM glutamine, 25 mM HEPES, 10% heat inactivated fetal bovine serum, 100 U/ml penicillin, 10 mg/ml streptomycin, 5 mM R-mercaptoethanol) in a concentration 1 million cells per milliliter. The assay was performed in 96-well plates in duplicates or triplicates, where each well was pre-coated with 3 μg/ml anti-CD3 antibody for 3-5 h at 37° C. Each well was loaded with 100,000 cells. Drugs were added to the wells at the relevant concentrations. The plate was incubated for 68 h at 37° C. (95% O₂, 5% CO₂) and then, a media-soluble tetrazolium dye MTT was added to a final concentration of 1 mM after which the plate was incubated for additional 4 h. The plate was then centrifuged at 2,000 RPM for 10 min to pellet the insoluble purple formazan crystals. The supernatant culture media was collected, stored at −80° C. and used for analysis of cytokines using the multiplex proximity extension assay (PEA). The formazan crystal pellet was dissolved in DMSO and the plate was read within 10 min using a Multiskan MS plate reader (Labsystems) at 550 nm. The optical density value was used as the proliferation index value. Drugs were purchased from Sigma-Aldrich or Tocris (Bristol, UK).

1.7. Multiplex PEA for Cytokine Measurements

Plasma samples, and culture media samples that were collected from plate wells at the end of the proliferation assay, were analyzed by multiplex PEA with an Olink Inflammation 196X96 panel, targeting 92 proteins related to inflammation (Olink Proteomics, Uppsala, Sweden) as previously described (Edvinsson et al., 2017, Assarsson et al., 2014, Larsson et al., 2015, Larssen et al., 2017). Briefly, 1 μl of sample (plasma samples or cell culture media samples) or negative control was mixed with 3 μl probe solution containing a set of 92 paires DNA-oligonucleotide-conjugated antibodies. Upon recognition of a target protein by a pair of probes, the DNA oligonucletodies on the antibodies are brought in proximity and hybridize to each other, followed by enzymatic DNA polymerization to form a new DNA molecule. The newly formed DNA molecule is then amplified and quantified using a microfluidic real-time qPCR, BioMark™ HD (Fluidigm, South San Francisco, Calif., USA). The generated quantification cycle (Cq) values are normalized against spiked-in controls to convert Cq values to Normalized Protein eXpression (NPX) value on log2 scale. NPX is an arbitrary unit, which is positively correlated to protein concentration. These NPX data were then converted to linear data, using the formula 2^{NPX}, prior to further statistical analysis. Limit of detection (LOD) for each protein was defined as three standard deviations above the background. Proteins with levels below LOD were excluded from further data analysis.

1.8. Total RNA Isolation and T Cell RNA Sequencing

Total RNA was extracted from T cells using Direct-zol™ RNA MicroPrep (Zymo Research, Irvine, Calif., USA) according to manufacturer's recommendation. cDNA libraries were prepared according to Smart-seq2 protocol (Picelli et al., 2013). For Illumina sequencing libraries, 2 ng of cDNA was fragmented, amplified (Picelli et al., 2014), pooled and sequenced on Illumina HiSeq 2500. Single-end 43 bp reads were generated and mapped to human reference genome GRCh38 by employing STAR (version 2.4.1) with parameter outSAMstrandField intronMotif (Dobin et al., 2013). Reads per kilobase transcript per million mapped reads (RPKM) from RefSeq gene annotations were calculated using RPKM for genes (Ramskold et al., 2009). The uniquely mapped reads were considered for the downstream analyses.

1.9. Statistical Analysis

Statistical analysis and data mining were performed using Statistica 12 (StatSoft Scandinavia, Uppsala, Sweden), GraphPad Prism 7 (La Jolla, Calif., USA) and edgeR bioconductor package. The statistical tests were performed after omitting outliers identified by the Tukey test. The differences between groups were assessed by nonparametric Kruskal-Wallis ANOVA on ranks with Dunn's post hoc test. The contingency of sex equality was accessed by Fisher's exact test. Comparison of demographic data between the two groups was based on a non-parametric Mann Whitney test for non-normally distributed data and a two-tailed Student's t-test for normally distributed. Normality of data was assessed by D'Agostino & Pearson omnibus normality test. The correlation between inflammatory cytokines and demographic factors was accessed using non-parametric Spearman rank correlation. The significance level was set to p<0.05.

2. Results

Demographic data for the ND individuals (n=30) and the individuals with T1D (n=64) that participated in the study is presented in Table S1. As expected the individuals with T1D had higher levels of fasting glucose and HbA1c (Table S1). In addition, the individuals with T1D were, on the average, slightly older and had a higher BMI (Table S1). The creatinine levels did not differ between the two groups but the glomerular filtration rate was higher in the diabetes group (Table S1). Islet autoantibodies (GAD and IA2) were not detected in any of the healthy individuals.

2.1. Cytokines in Plasma from ND and T1D Individuals.

Immune cells secrete a large number of small proteins, collectively termed cytokines, which may have a protective function or act as pro-inflammatory molecules. We investigated whether the types of cytokines in plasma differed between ND individuals and T1D subjects. We used the multiplex PEA to measure the blood levels of a panel of 92 cytokines that are most commonly associated with inflammation (http://www.olink.com/products/inflammation/#). The assay that uses paired cytokine-specific antibodies for the different cytokines allows comparison of the levels of the same cytokine in samples from e.g. ND individuals and T1D subjects. However, the assay format does not support comparison of the absolute levels of one cytokine to another as the affinities of the antibodies for their cognate targets may vary. As illustrated in FIG. 2a, 73 out of 92 analyzed cytokines were detected in plasma from the donors, of which 26 cytokines were significantly up-regulated and only one cytokine, FGF-21 down-regulated in plasma from T1D subjects as compared to ND individuals (FIG. 2b).

We then examined if the neurotransmitter GABA varied in concentration in plasma between the ND and T1D individuals (FIG. 2c). The GABA concentration range was similar for the two groups but there was a trend for increased plasma GABA concentration in T1D subjects resulting in a significantly higher average concentration in the T1D group (ND: 501±32 nM; T1D: 649±42 nM; p<0.05). No correlation was observed for GABA concentrations with age or disease duration. In contrast, when the concentration of GABA was correlated with cytokines detected in plasma, levels of 10 cytokines were significantly correlated (p<0.05) with the plasma GABA concentration (FIG. 2d; Table S3).

TABLE S3
Cytokines and GABA concentration
Significant Spearman rank correlation between cytokines
and GABA concentration in plasma

| Cytokines | R values | P values |
|---|---|---|
| CASP-8 | 0.3616 | 0.03 |
| EN-RAGE | −0.4463 | 0.0082 |
| IL10 | 0.364 | 0.025 |
| PD-L1 | 0.9801 | 0.0001 |
| TNF-B | 0.4846 | 0.0018 |
| STAMPB | 0.383 | 0.016 |
| IL-10RB | 0.4427 | 0.0048 |
| HGF | 0.4403 | 0.005 |
| TRAIL | 0.4188 | 0.008 |
| Flt3L | 0.3604 | 0.034 |

Samples were combined from nondiabetic and type 1 diabetic individuals.

2.2. GABA Inhibits Proliferation of PBMCs from T1D Subjects and Responder CD4⁺ T Cells.

To further examine the effects of GABA on the immune cells, we stimulated PBMCs and CD4⁺ T cells with anti-CD3 antibody to induce proliferation of CD3⁻ positive T cells. We then examined effects on proliferation of GABA and the GABAA antagonist, picrotoxin. In PBMCs from ND individuals, GABA did not inhibit proliferation of the cells (FIG. 3a). In contrast, cell proliferation of PBMCs from T1D subjects was inhibited in the presence of 100 nM GABA by approximately 20%, while GABA in concentration of 1 μM did not have any inhibitory effect on the cell proliferation. The inhibition was reversed by picrotoxin (FIG. 3b). We have previously demonstrated that GABA may inhibit T cells proliferation (Bjurstom et al., 2008) and, thus, examined whether CD4⁺ T cells from ND individuals varied in their sensitivity to GABA. The results revealed that CD4⁺ T cells from the ND individuals could be grouped into responders (n=15) and non-responders (n=7) depending on whether or not GABA affected the proliferation of the cells (FIG. 3c). We examined further if GABA, in a dose-dependent manner, could regulate proliferation of the responder CD4+ T cells (FIG. 3d). Application of different GABA concentrations demonstrated increased inhibition of proliferation for 1 to 500 nM GABA with a maximum inhibition of approximately 40% by 500 nM GABA, whereas no inhibition of proliferation of the cells was recorded in 1 μM GABA (FIG. 3d). This is in line with the fact that saturating GABA concentrations cause desensitization and thus, non-functionality of GABA_{A} receptors. The open channel blocker picrotoxin partially reversed the 500 nM GABA-induced inhibition (FIG. 3e) and the GABAA agonists muscimol (100 nM) and TACA (100 nM) inhibited the proliferation similar to GABA (FIG. 3f), whereas the GABA_{B} antagonist CGP52432 (50 μM) and the GABA_{B} agonist baclofen (100 nM) were ineffective (FIG. 3e, f). Together the results highlight that immune cells and in particular CD4⁺ T cells can be divided into subgroups depending on whether or not their proliferation is regulated by GABA. The greater inhibition observed in the PBMCs from the T1D subjects as compared to ND individuals is consistent with the presence of expanded T cells clones that respond to GABA in T1D (Tong et al., 2016).

GABA can potentially activate GABA_{A} and GABA_{B} receptors in the immune cells (Tian et al., 2004, Bjurstom et al., 2008, Bhandage et al., 2015). We, therefore, measured the expression level of the GABAA and GABAB receptor subunits in PBMCs. The most prominent GABAA receptor subunit was the p2 that was similarly expressed and present in most samples from both ND and T1D individuals (FIG. 3g, Table S4). Protein expression of p2 was confirmed by western blot analysis (FIG. 8). Of the two GABA_{B} receptor subunits, only the GABA_{B}R1 was commonly expressed. Our results confirmed previously reported expression of GABA receptors in PBMCs (Bhandage et al., 2015). We further found that the mRNA expression levels of auxiliary proteins of GABA_{A} receptors gephyrin and GABARAP were similar but the GABA transporters GAT3, BGT1 and the enzyme GABA-T plus the insulin receptor were significantly increased, whereas the expression level of radixin decreased in PBMCs from T1D individuals (FIG. 9 Table S4). Expression of Cl⁻ transporters was altered in T1D (FIG. 3h). The transporter that moves Cl⁻ into the cell, NKCC1, was significantly down-regulated, whereas the transporters that move Cl⁻ out of the cells, KCC3 and KCC4, were up-regulated in PBMCs from T1D subjects. Protein expression of NKCC1 was confirmed by western blot analysis (FIG. 8). Since the effects of GABA_{A} receptors are related to the Cl⁻ equilibrium potential in the cells, any changes in intracellular chloride will have consequences for GABA_{A} signaling. We further investigated if GABA in submicromolar (100 or 500 nM) concentrations activated single-channel currents in the T cells. GABA activated GABA_{A} single-channel currents in the cells (n=54), which were blocked by picrotoxin. The GABA_{A} receptors conductance ranged from 9 to 45 pS (FIG. 3i).

TABLE S4
GABA signaling-system proteins mRNA expression

| | |
|---|---|
| Number of samples | |
| expressed out of S4 | Percentage |

GABA_{A} receptor subunits

| | | |
|---|---|---|
| GABRA1 (α1) | 0 | 0 |
| GABRA2 (α2) | 0 | 0 |
| GABRA3 (α3) | 2 | 3.7 |
| GABRA4 (α4) | 1 | 1.9 |
| GABRA5 (α5) | 1 | 1.9 |
| GABRA6 (α6) | 10 | 18.5 |
| GABRB1 (β1) | 6 | 11.1 |
| GABRB2 (β2) | 19 | 35.2 |
| GABRB3 (β3) | 0 | 0.0 |
| GABRG1 (γ1) | 0 | 0.0 |
| GABRG2 (γ2) | 1 | 1.9 |
| GABRG3 (γ3) | 0 | 0.0 |
| GABRD (δ) | 11 | 20.4 |
| GABRE (ε) | 11 | 20.4 |
| GABRQ (θ) | 1 | 1.9 |
| GABRP (η) | 0 | 0.0 |
| GABRR1 (ρ1) | 0 | 0.0 |
| GABRR2 (ρ2) | 52 | 96.3 |
| GABRR3 (ρ3) | 2 | 3.7 |

GABAₑ receptor subunits

| | | |
|---|---|---|
| GABRR1 (GABA-B1) | 54 | 100.0 |
| GABRR2 (GABA-B2) | 1 | 1.9 |

Chloride transporter

| | | |
|---|---|---|
| SLC12A2 (NKCC1) | 50 | 92.6 |
| SLC12A1 (NKCC2) | 0 | 0 |
| SLC12A4 (KCC1) | 53 | 98.1 |
| SLC12A5 (KCC2) | 2 | 3.7 |
| SLC12A6 (KCC3) | 53 | 98.1 |
| SLC12A7 (KCC4) | 53 | 98.1 |

GABA_{A} receptor accessory proteins

| | | |
|---|---|---|
| ABAT (GABA-T) | 54 | 100.0 |
| GABARAP | 53 | 98.1 |
| Gephyrin | 51 | 94.4 |
| Radixin | 53 | 98.1 |
| GAT1 | 0 | 0.0 |
| GAT2 | 10 | 18.5 |
| GAT3 | 25 | 46.3 |
| BGT1 | 52 | 96.3 |
| GAD65 | 0 | 0.0 |
| GAD67 | 4 | 7.4 |

Islet hormone receptor

| | | |
|---|---|---|
| Insulin receptor | 52 | 96.3 |

Total of 54 samples were examined, 21 nondiabetic and 33 type 1 diabetic PBMCs

2.3. Cholesterol Biosynthesis Gene Levels are Regulated in T cells from T1D Subjects.

We applied RNA-seq to examine the transcriptome of isolated CD3+ T cells from ND individuals and T1D subjects (FIG. 3j). A total of 16,684 genes were identified after passing the quality control and deposited at GEO database (https://www.ncbi.nlm.nih.gov/geo/). To reduce the bias of genes with low counts and only expressed in a few samples, we used the dataset with 8,669 genes that are expressed in more than ⅓ of the samples (RPKM>5) in both T1D subjects and ND individuals. Eleven genes (edgeR p<0.05, FDR<0.05) were differentially expressed by more than two-fold between T1D and ND samples, of which only 1 gene (SCARNA21) was up-regulated and 10 genes were down-regulated in samples from T1D subjects (FIG. 3j). Among the down-regulated genes, 5 genes (SQLE, MSMO1, DHCR24, CYP51A1, HMGCS1) are involved in the cholesterol biosynthesis pathway and 1 gene (INSIG1) encodes an endoplasmic reticulum protein that regulates cholesterol biosynthesis (REACTOME, http://reactome.org) (Table S5). Interestingly, the expression levels of MSMO1 and CYP51A1 were significantly correlated with the plasma GABA concentration (p<0.05) (FIG. 10). Furthermore, the expression of MSMO1 was also significantly correlated with BMI, fasting glucose and HbA1c levels (p<0.05) (FIG. 10).

TABLE S5
The list of differentially expressed genes between type 1 diabetic (n = 31)
and nondiabetic (n = 18) individuals analyzed by EdgeR

| | | Log2 (Fold | | |
|---|---|---|---|---|
| Gene Symbol | Gene name | Change) | P value | FDR |
| PSAT1 | Phosphoserine aminotransferase | −2.046572497 | 2.88689E−07 | 0.001477106 |
| MTRNR2LB | MT-RNR2-like 8 | −3.702230773 | 3.40779E−07 | 0.001477106 |
| SQLE | Squalene monooxygenase | −2.006525166 | 1.38982E−06 | 0.004016125 |
| MSMO1 | Methylsterol monooxygenase 1 | −1.606541543 | 2.40393E−06 | 0.004409674 |
| DHCR24 | Delta(24)-sterol reductase | −1.987818017 | 2.54336E−06 | 0.004409674 |
| CYPS1A1 | Lanosterol 14-alpha demethylase | −1.679899788 | 1.09233E−05 | 0.051782366 |
| AARS | Alanine-tRNA ligase, cytoplasmic | −1.027073283 | 1.32314E−05 | 0.016388168 |
| LDLR | Low density lipoprotein receptor | −1.739864323 | 1.55876E−05 | 0.016891142 |
| INSIG1 | Insulin induced gene 1 | −1.170142188 | 2.68484E−05 | 0.025861984 |
| MUT | Methylmalonyl-CoA mutase | −0.828339766 | 4.16762E−05 | 0.03224786 |
| STAT1 | Signal transducer and activator of transcription 1 | −0.833595514 | 4.29821E−05 | 0.03224786 |
| SCARNA21 | Small Cajal body-specific RNA 21 | 1.164498735 | 4.75355E−05 | 0.03224786 |
| ZNF181 | Zinc finger protein 161 | −0.842904195 | 4.83588E−05 | 0.03224786 |
| TARS | Threonyl-tRNA synthetase | −0.763887081 | 5.31431E−05 | 0.032908866 |
| IDI1 | isopentenyl-diphosphate delta isomerase 1 | −0.89363201 | 6.22089E−05 | 0.038952573 |
| HMGCS1 | 3-hydroxy-3-methylglutaryl-CoA synthase 1 | −1.132812904 | 7.53878E−05 | 0.040646048 |

Quality control was conducted by the following criteria:
(1) read counts for each sample were over 500,000;
(2) The total and uniquely mapping ratio of each sample were more than 70% and 40%, respectively;
(3) The filtered genes were expressed in at least 1/3 samples from both type 1 diabetic and nondiabetic donors with RPKM > 5;
(4) The Spearman correlation coefficient with any two samples was over 0.4;
(5) Each sample expressed at least 5000 genes (RPKM > 6).
FRD: false discovery rate.

2.4. GABA Regulates Release of Pro- and Anti-Inflammatory Cytokines from PBMCs.

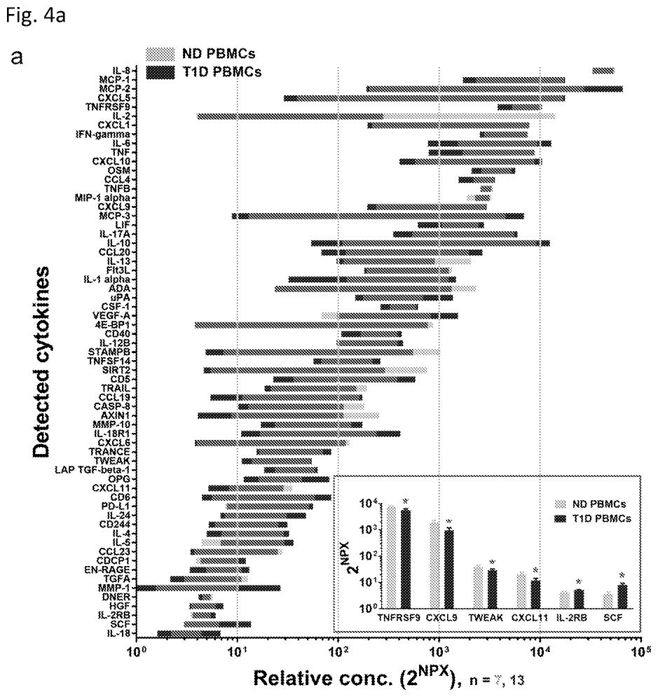

It is possible that GABA signalling regulates what cytokines are released from the immune cells. We, therefore, examined the culture media from the anti-CD3 stimulated PBMCs using the inflammatory related protein panel described above to study which of the 92 cytokines are released by the cells and whether GABA affects secretion of specific cytokines. FIG. 4a shows the levels of the different cytokines detected in the culture media harboring proliferating cells from ND individuals and T1D subjects. In the absence of GABA, a difference in the secretion level was observed for six cytokines (FIG. 4a insert). Somewhat surprisingly, the results shown in FIG. 4b and FIG. 4c and also Table S6 and S7 demonstrate that GABA regulated the release of many pro- and anti-inflammatory cytokines. In the culture media from stimulated PBMCs, GABA (100 nM) significantly inhibited release of 16 cytokines from ND individuals (n=7; FIGS. 4b) and 47 cytokines from T1D subjects (n=13; FIG. 4c), respectively, and, additionally, increased the release of two cytokines from T1D subjects (FIG. 4c). Cytokines released by stimulated PBMCs, which were affected by GABA can be grouped according to their function (FIG. 4d). The group of cytokines with the largest difference between ND and T1D PBMCs was the one associated with chemotaxis. The results are in agreement with that GABA regulates release of cytokines from PBMCs in both health and in disease.

TABLE S6
GABA inhibits secretion of cytokines from ND PBMCs

| Nondiabetic | | | |
|---|---|---|---|
| PBMCs | GABA 100 nM | | |
| Cytokines | % Mean | SEM | P values |
| TNFB | 95.7 | 3.1 | 0.0250 |
| CCL4 | 95.3 | 5.1 | 0.0250 |
| CD244 | 92.5 | 5.4 | 0.0250 |
| IL-18R1 | 90.7 | 7.5 | 0.0250 |
| TNFRSF9 | 99.6 | 4.9 | 0.0250 |
| TNFSF14 | 89.2 | 5.5 | 0.0250 |
| LIF | 88.5 | 5.4 | 0.0250 |
| OSM | 88.1 | 4.8 | 0.0250 |
| PD-L1 | 87.8 | 6.7 | 0.0460 |
| TWEAK | 86.3 | 7.6 | 0.0250 |
| IL-12B | 84.8 | 6.3 | 0.0250 |
| AXIN1 | 82.3 | 7.4 | 0.0210 |
| OPG | 81.8 | 6.6 | 0.0250 |
| SIRT2 | 81.6 | 9.7 | 0.0250 |
| CXCL9 | 81.3 | 11.0 | 0.0250 |
| TNF | 74.1 | 8.8 | 0.0250 |

Cytokines reissued into the culture medium by PBMCs from nondiabetic (ND) individuals.
GABA significantly altered levels of the cytokines.

TABLE S7
GABA inhibits secretion of cytokines from T1D PBMCS

| Type 1 diabetic | | | |
|---|---|---|---|
| PBMCs | GABA 100 nM | | |
| Cytokines | % Mean | SEM | P values |
| IL-4 | 117.0 | 11.0 | 0.0060 |
| MIP-1 alpha | 106.2 | 1.8 | 0.0027 |
| IL-24 | 86.9 | 5.8 | 0.0047 |
| CCL4 | 85.1 | 2.8 | 0.0025 |
| VEGF-A | 82.1 | 4.2 | 0.0012 |
| MMP-1 | 78.4 | 6.8 | 0.0048 |
| CD244 | 76.3 | 5.6 | 0.0130 |
| IL-2RB | 73.0 | 3.4 | 0.0005 |
| EN-RAGE | 71.1 | 4.1 | 0.0010 |
| TNFRSF9 | 71.1 | 4.9 | 0.0001 |
| CSF-1 | 70.5 | 4.5 | 0.0001 |
| LIF | 70.0 | 8.7 | 0.0100 |
| TNFSF14 | 69.9 | 5.5 | 0.0002 |
| OSM | 69.8 | 7.5 | 0.0002 |
| LAP TGF-beta-1 | 67.6 | 3.7 | 0.0001 |
| TRAIL | 67.0 | 6.7 | 0.0008 |
| μPA | 86.3 | 5.1 | 0.0001 |
| IL-5 | 85.3 | 4.6 | 0.0001 |
| IL-18R1 | 63.3 | 10.1 | 0.0092 |
| Flt3L | 62.9 | 4.0 | 0.0001 |
| AXIN1 | 62.8 | 6.4 | 0.0018 |
| CXCL11 | 61.4 | 6.5 | 0.0000 |
| MMP-10 | 61.3 | 5.4 | 0.0001 |
| TGFA | 61.1 | 3.1 | 0.0001 |
| CASP-8 | 60.9 | 3.6 | 0.0001 |
| CXCL5 | 68.1 | 7.0 | 0.0001 |
| IL-17A | 58.9 | 7.0 | 0.0001 |
| PD-L1 | 57.0 | 3.5 | 0.0014 |
| TWEAK | 55.7 | 4.5 | 0.0001 |
| CXCL6 | 54.0 | 8.9 | 0.0001 |
| STAMP8 | 53.9 | 5.5 | 0.0001 |
| SIRT2 | 53.5 | 9.1 | 0.0052 |
| ADA | 53.4 | 4.4 | 0.0001 |
| IL-12B | 53.3 | 5.5 | 0.0001 |
| IL-13 | 53.0 | 4.9 | 0.0001 |
| MCP-1 | 53.0 | 7.9 | 0.0002 |
| CXCL1 | 52.6 | 7.6 | 0.0001 |
| IL-10 | 61.4 | 6.7 | 0.0001 |
| IL-6 | 49.4 | 5.8 | 0.0001 |
| 4E-BP1 | 45.5 | 4.3 | 0.0001 |
| TNF | 47.4 | 6.6 | 0.0001 |
| CXCL9 | 45.5 | 10.0 | 0.0001 |
| OPG | 45.1 | 5.0 | 0.0001 |
| MCP-3 | 44.6 | 8.6 | 0.0001 |
| CCL19 | 43.8 | 7.6 | 0.0001 |
| IL-1 alpha | 43.5 | 6.6 | 0.0001 |
| CXCL10 | 42.5 | 5.6 | 0.0001 |
| MCP-2 | 35.4 | 5.9 | 0.0001 |
| CCL20 | 31.5 | 6.8 | 0.0001 |

Cytokines released into culture medium by PBMCs, from type 1 diabetic (T1D) patients.
GABA significantly altered levels of the cytokines.

2.5. GABA Regulates Release of Pro- and Anti-Inflammatory Cytokines from CD4⁺ T Cells.

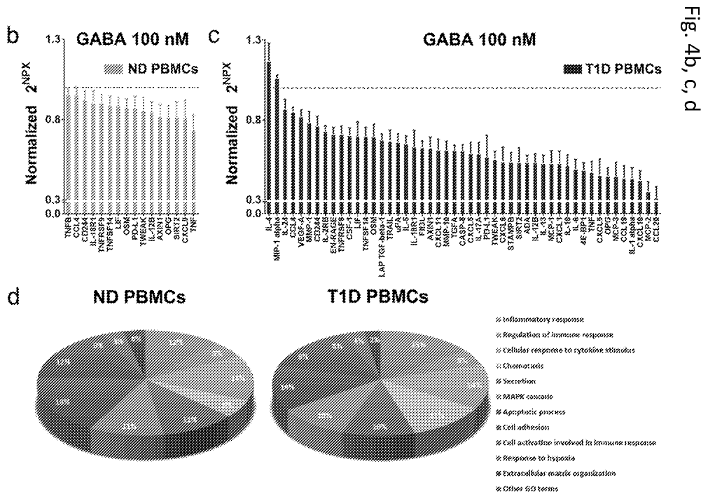

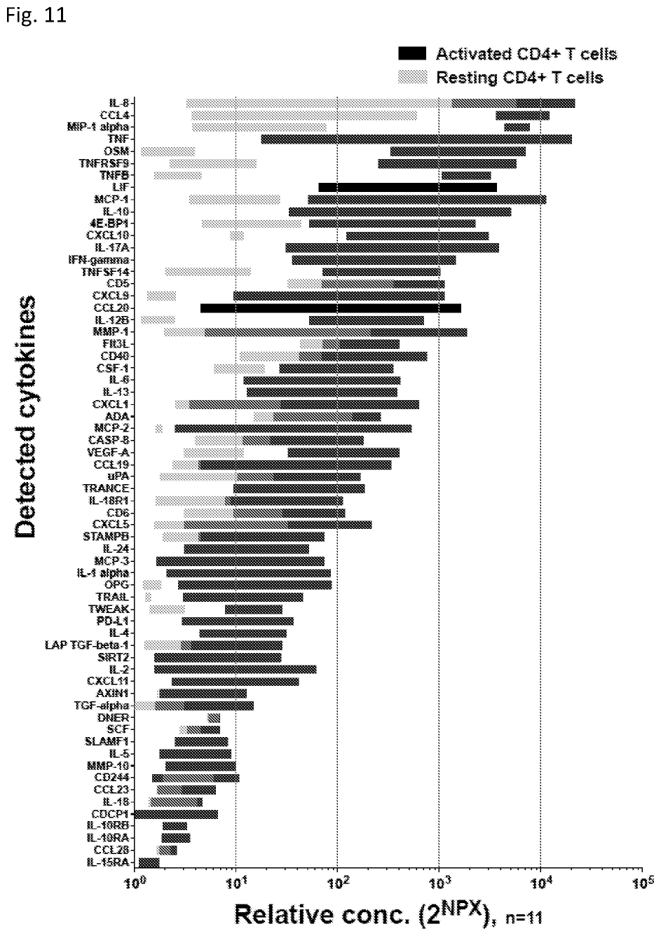

ND individuals could be divided into two groups based on whether or not their stimulated CD4⁺ cells responded to GABA in the proliferation assay (see FIG. 3c). We termed the two groups of stimulated CD4+ cells, responder (n=15) and non-responder (n=7) T cells. Stimulated T cells normally release more and higher levels of cytokines than resting T cells (FIG. 11). We examined if CD4⁺ T cells from responders and non-responders differentially secreted cytokines and then, if the release in the two groups were affected by GABA. FIG. 5a shows that upon stimulation, cells from both groups released several cytokines and to similar levels. Only levels of three cytokines were significantly different between the two groups (FIG. 5a insert). In contrast regulation of cytokine release by GABA was far more prominent in responder as compared to non-responder T cells. In non-responder T cells, GABA at 100 and 500 nM concentration regulated secretion of only four cytokines at each concentration (FIG. 5b, c; Table S8). This is in contrast to the responder T cells where GABA significantly inhibited release of many cytokines and, interestingly, the different concentrations of GABA inhibited release of somewhat different cytokines (FIG. 5b, c; Table S9). In the presence of 100 nM GABA, release of 27 cytokines were significantly decreased as compared to 25 cytokines in the presence of 500 nM GABA. Picrotoxin reversed the effects of GABA. Of these cytokines, secretion of 15 cytokines, including both Th1- and Th2-type cytokines e.g. TNF-α and IL-13, were inhibited by both 100 and 500 nM GABA (FIG. 6b). In the presence of 100 nM GABA, another 12 distinct cytokines, including the Th2-type IL-6 and IL-24 cytokines, were specifically inhibited. Inhibition of the Th1-type cytokines INF-γ and TNF-β plus the Th2-type cytokine IL-5 was observed only when GABA was present at 500 nM concentration. FIG. 5d shows that the proportion of cytokines associated with chemotaxis remained similar to what was determined for PBMCs from T1D subjects. However, when the GABA concentration was increased from 100 to 500 nM, the proportion of cytokines associated with secretion and MAPK decreased, whereas those associated with cellular response to cytokine stimulus and regulation of immune response increased. The results demonstrate that GABA in a concentration-dependent manner regulates cytokine secretion from CD4⁺ T cells.

TABLE S8
Non-responder CD4⁺ T cells

| Non-responder | | | |
|---|---|---|---|
| CD4⁺ T cells | GABA 100 nM | | |
| Cytokines | % Mean | SEM | P values |
| MMP-1 | 124.2 | 13.4 | 0.004 |
| IL-1 alpha | 112.8 | 3.6 | 0.0062 |
| MCP-3 | 112.5 | 3.3 | 0.021 |
| CASP-8 | 112.0 | 3.5 | 0.004 |
| Non-responder | | | |
| CD4⁺ T cells | GABA 500 nM | | |
| Cytokines | % Mean | SEM | P values |
| PD-L1 | 120.4 | 12.0 | 0.009 |
| IL-6 | 111.8 | 1.8 | 0.009 |
| TGF-alpha | 109.5 | 1.9 | 0.004 |
| IL-5 | 87.2 | 4.1 | 0.009 |

Cytokines released into the culture medium by CD4⁺ T cells.
GABA significantly altered levels of the cytokines.

TABLE S9
Responder CD4⁺ T cells

| Responder CD4⁺ | | | |
|---|---|---|---|
| T cells | GABA 100 nM | | |
| Cytokines | % Mean | SEM | P values |
| DNER | 94.6 | 2.4 | 0.014 |
| TRANCE | 89.3 | 3.1 | 0.014 |
| TNFRSF9 | 86.3 | 2.5 | 0.014 |
| CSF-1 | 84.2 | 6.5 | 0.014 |
| LAP TGF-beta-1 | 79.6 | 7.6 | 0.014 |
| Flt3L | 79.3 | 5.6 | 0.014 |
| ADA | 79.3 | 5.7 | 0.014 |
| MCP-1 | 76.9 | 7.8 | 0.014 |
| μPA | 78.9 | 9.7 | 0.014 |
| OSM | 76.4 | 11.1 | 0.014 |
| IL-18R1 | 76.1 | 6.4 | 0.014 |
| TRAIL | 75.9 | 7.8 | 0.014 |
| IL-24 | 71.8 | 4.2 | 0.021 |
| IL-6 | 71.6 | 13.1 | 0.014 |
| TNFSF14 | 70.6 | 8.0 | 0.014 |
| OPG | 70.5 | 12.4 | 0.014 |
| IL-13 | 69.9 | 9.8 | 0.014 |
| CXCL11 | 69.1 | 11.3 | 0.014 |
| STAMPB | 68.6 | 6.5 | 0.014 |
| CCL19 | 68.7 | 11.1 | 0.014 |
| 4E-BP1 | 67.1 | 10.0 | 0.014 |
| LIF | 66.9 | 13.1 | 0.014 |
| CASP-8 | 66.3 | 6.0 | 0.014 |
| CXCL10 | 62.7 | 17.7 | 0.014 |
| CCL20 | 61.5 | 9.1 | 0.014 |
| CSCP1 | 60.6 | 10.8 | 0.025 |
| TNF | 55.4 | 11.1 | 0.014 |
| Responder CD4⁺ | | | |
| T cells | GABA 500 nM | | |
| Cytokines | % Mean | SEM | P values |
| TNFB | 69.0 | 6.2 | 0.009 |
| DNER | 68.6 | 6.1 | 0.009 |
| SCF | 61.8 | 6.9 | 0.009 |
| IL-8 | 81.2 | 12.7 | 0.009 |
| CD40 | 77.8 | 9.2 | 0.009 |
| IL-5 | 76.0 | 12.3 | 0.014 |
| IFM-gamma | 73.2 | 11.6 | 0.009 |
| CXCL1 | 72.2 | 12.1 | 0.009 |
| IL-12B | 69.4 | 14.8 | 0.009 |
| μPA | 68.0 | 13.7 | 0.009 |
| CXCL11 | 66.6 | 13.2 | 0.009 |
| CXCL5 | 66.0 | 9.9 | 0.009 |
| IL-18R1 | 85.8 | 10.6 | 0.009 |
| IL-13 | 83.0 | 14.3 | 0.009 |
| CXCL9 | 62.2 | 14.3 | 0.009 |
| CASP-8 | 61.4 | 13.1 | 0.009 |
| TNFSF14 | 60.0 | 12.8 | 0.009 |
| CXCL10 | 60.6 | 16.2 | 0.009 |
| STAMPB | 57.8 | 13.4 | 0.009 |
| CCL20 | 66.0 | 13.7 | 0.009 |
| OPG | 66.4 | 13.3 | 0.009 |
| 4E-BP1 | 50.6 | 11.8 | 0.009 |
| CCL19 | 59.9 | 11.7 | 0.009 |
| TNF | 47.8 | 14.4 | 0.009 |
| CDCP1 | 44.3 | 14.5 | 0.025 |

Cytokines released into culture medium by CD4⁺ T cells.
GABA significantly altered levels of the cytokines.

### REFERENCES

All references cited herein are expressly incorporated by reference in their entirety.

ABU SHMAIS, G. A., AL-AYADHI, L. Y., AL-DBASS, A. M. & EL-ANSARY, A. K. 2012. Mechanism of nitrogen metabolism-related parameters and enzyme activities in the pathophysiology of autism. J Neurodev Disord, 4, 4.

ALAM, S., LAUGHTON, D. L., WALDING, A. & WOLSTENHOLME, A. J. 2006. Human peripheral blood mononuclear cells express GABAA receptor subunits. Mol Immunol, 43, 1432-42.

ASSARSSON, E., LUNDBERG, M., HOLMQUIST, G., BJORKESTEN, J., THORSEN, S. B., EKMAN, D., ERIKSSON, A., RENNEL DICKENS, E., OHLSSON, S., EDFELDT, G., ANDERSSON, A. C., LINDSTEDT, P., STENVANG, J., GULLBERG, M. & FREDRIKSSON, S. 2014. Homogenous 96-plex PEA immunoassay exhibiting high sensitivity, specificity, and excellent scalability. PLoS One, 9, e95192.

BAEKKESKOV, S., AANSTOOT, H. J., CHRISTGAU, S., REETZ, A., SOLIMENA, M., CASCALHO, M., FOLLI, F., RICHTER-OLESEN, H. & D E CAMILLI, P. 1990. Identification of the 64K autoantigen in insulin-dependent diabetes as the GABAsynthesizing enzyme glutamic acid decarboxylase. Nature, 347, 151-6.

BAEKKESKOV, S., HUBBELL, J. A. & PHELPS, E. A. 2017. Bioengineering strategies for inducing tolerance in autoimmune diabetes. Adv Drug Deliv Rev, 114, 256-265.

BARRAGAN, A., WEIDNER, J. M., JIN, Z., KORPI, E. R. & BIRNIR, B. 2015. GABAergic signalling in the immune system. Acta Physiol (Oxf), 213, 819-27.

BHANDAGE, A. K., HELLGREN, C., JIN, Z., OLAFSSON, E. B., SUNDSTROM-POROMAA, I. & BIRNIR, B. 2015. Expression of GABA receptors subunits in peripheral blood mononuclear cells is gender dependent, altered in pregnancy and modified by mental health. Acta Physiol (Oxf), 213, 575-85.

BHANDAGE, A. K., JIN, Z., HELLGREN, C., KOROL, S. V., NOWAK, K., WILLIAMSSON, L., SUNDSTROM-POROMAA, I. & BIRNIR, B. 2017. AMPA, NMDA and kainate glutamate receptor subunits are expressed in human peripheral blood mononuclear cells (PBMCs) where the expression of GluK4 is altered by pregnancy and GluN2D by depression in pregnant women. J Neuroimmunol, 305, 51-58.

BHAT, R., AXTELL, R., MITRA, A., MIRANDA, M., LOCK, C., TSIEN, R. W. & STEINMAN, L. 2010. Inhibitory role for GABA in autoimmune inflammation. Proc Natl Acad Sci U S A, 107, 2580-5.

BIRNIR, B. & KORPI, E. R. 2007. The impact of sub-cellular location and intracellular neuronal proteins on properties of GABAA receptors. Curr Pharm Des, 13, 3169-77.

BJURSTOM, H., WANG, J., ERICSSON, I., BENGTSSON, M., LIU, Y., KUMAR-MENDU, S., ISSAZADEH-NAVIKAS, S. & BIRNIR, B. 2008. GABA, a natural immunomodulator of T lymphocytes. J Neuroimmunol, 205, 44-50.

BU, D. F., ERLANDER, M. G., HITZ, B. C., TILLAKARATNE, N. J., KAUFMAN, D. L., WAGNER-MCPHERSON, C. B., EVANS, G. A. & TOBIN, A. J. 1992. Two human glutamate decarboxylases, 65-kDa GAD and 67-kDa GAD, are each encoded by a single gene. Proc Natl Acad Sci U S A, 89, 2115-9.

CAICEDO, A. 2013. Paracrine and autocrine interactions in the human islet: more than meets the eye. Semin Cell Dev Biol, 24, 11-21.

DIONISIO, L., JOSE DE ROSA, M., BOUZAT, C. & ESANDI MDEL, C. 2011. An intrinsic GABAergic system in human lymphocytes. Neuropharmacology, 60, 513-9.

DOBIN, A., DAVIS, C. A., SCHLESINGER, F., DRENKOW, J., ZALESKI, C., JHA, S., BATUT, P., CHAISSON, M. & GINGERAS, T. R. 2013. STAR: ultrafast universal RNAseq aligner. Bioinformatics, 29, 15-21.

DUTHEY, B., HUBNER, A., DIEHL, S., BOEHNCKE, S., PFEFFER, J. & BOEHNCKE, W. H. 2010. Anti-inflammatory effects of the GABAB receptor agonist baclofen in allergic contact dermatitis. Exp Dermatol, 19, 661-6.

EDVINSSON, A., BRANN, E., HELLGREN, C., FREYHULT, E., WHITE, R., KAMALIMOGHADDAM, M., OLIVIER, J., BERGQUIST, J., BOSTROM, A. E., SCHIOTH, H. B., SKALKIDOU, A., CUNNINGHAM, J. L. & SUNDSTROM-POROMAA, I. 2017. Lower inflammatory markers in women with antenatal depression brings the M1/M2 balance into focus from a new direction.

Psychoneuroendocrinology, 80, 15-25.

EL-ANSARY, A. K., BACHA, A. B. & AYAHDI, L. Y. 2011. Relationship between chronic lead toxicity and plasma neurotransmitters in autistic subjects from Saudi Arabia. Clin Biochem, 44, 1116-20.

FARRANT, M. & NUSSER, Z. 2005. Variations on an inhibitory theme: phasic and tonic activation of GABAA receptors. Nat Rev Neurosci, 6, 215-29.

FIORINA, P. 2013. GABAergic system in beta-cells: from autoimmunity target to regeneration tool. Diabetes, 62, 3674-6.

FRANCO, R., PACHECO, R., LLUIS, C., AHERN, G. P. & O′CONNELL, P. J. 2007. The emergence of neurotransmitters as immune modulators. Trends Immunol, 28, 400-7.

FUKS, J. M., ARRIGHI, R. B., WEIDNER, J. M., KUMAR MENDU, S., JIN, Z., WALLIN, R. P., RETHI, B., BIRNIR, B. & BARRAGAN, A. 2012. GABAergic signaling is linked to a hypermigratory phenotype in dendritic cells infected by Toxoplasma gondii. PloS Pathog, 8, e1003051.

GASSMANN, M., SHABAN, H., VIGOT, R., SANSIG, G., HALLER, C., BARBIERI, S., HUMEAU, Y., SCHULER, V., MULLER, M., KINZEL, B., KLEBS, K., SCHMUTZ, M., FROESTL, W., HEID, J., KELLY, P. H., GENTRY, C., JATON, A. L., VAN DER PUTTEN, H., MOMBEREAU, C., LECOURTIER, L., MOSBACHER, J., CRYAN, J. F., FRITSCHY, J. M., LUTHI, A., KAUPMANN, K. & BETTLER, B. 2004. Redistribution of GABAB1 protein and atypical GABAB responses in GABAB2-deficient mice. J Neurosci, 24, 6086-97.

GOYETTE, J. & GAUS, K. 2017. Mechanisms of protein nanoscale clustering. Curr Opin Cell Biol, 44, 86-92.

HUBLER, M. J. & KENNEDY, A. J. 2016. Role of lipids in the metabolism and activation of immune cells. J Nutr Biochem, 34, 1-7.

JIN, Z., JIN, Y. & BIRNIR, B. 2011a. GABA-activated single-channel and tonic currents in rat brain slices. J Vis Exp.

JIN, Z., MENDU, S. K. & BIRNIR, B. 2011b. GABA is an effective immunomodulatory molecule. Amino Acids, 45, 87-94.

JOHNSTON, G. A., CHEBIB, M., HANRAHAN, J. R. & MEWETT, K. N. 2010. Neurochemicals for the investigation of GABAC receptors. Neurochem Res, 35, 1970-7.

KANAANI, J., CIANCIARUSO, C., PHELPS, E. A., PASQUIER, M., BRIOUDES, E., BILLESTRUP, N. & BAEKKESKOV, S. 2015. Compartmentalization of GABA Synthesis by GAD67 Differs between Pancreatic Beta Cells and Neurons. PLoS One, 10, e0117130.

KIDANI, Y. & BENSINGER, S. J. 2016. Modulating Cholesterol Homeostasis to Build a Better T Cell. Cell Metab, 23, 963-964.

KRETH, S., HEYN, J., GRAU, S., KRETZSCHMAR, H. A., EGENSPERGER, R. & KRETH, F. W. 2010. Identification of valid endogenous control genes for determining gene expression in human glioma. Neuro Oncol, 12, 570-9.

LARSSEN, P., WIK, L., CZARNEWSKI, P., ELDH, M., LOF, L., RONQUIST, K. G., DUBOIS, L., FREYHULT, E., GALLANT, C. J., OELRICH, J., LARSSON, A., RONQUIST, G., VILLABLANCA, E. J., LANDEGREN, U., GABRIELSSON, S. & KAMALI-MOGHADDAM, M. 2017. Tracing Cellular Origin of Human Exosomes Using Multiplex Proximity Extension Assays. Mol Cell Proteomics, 16, 502-511.

LARSSON, A., CARLSSON, L., GORDH, T., LIND, A. L., THULIN, M. & KAMALIMOGHADDAM, M. 2015. The effects of age and gender on plasma levels of 63 cytokines. J Immunol Methods, 425, 58-61.

LEDDEROSE, C., HEYN, J., LIMBECK, E. & KRETH, S. 2011. Selection of reliable reference genes for quantitative real-time PCR in human T cells and neutrophils. BMC Res Notes, 4, 427.

LEE, M., SCHWAB, C. & MCGEER, P. L. 2011. Astrocytes are GABAergic cells that modulate microglial activity. Glia, 59, 152-65.

LEVITE, M. 2008. Neurotransmitters activate T-cells and elicit crucial functions via neurotransmitter receptors. Curr Opin Pharmacol, 8, 460-71.

LEVITE, M., CHOWERS, Y., GANOR, Y., BESSER, M., HERSHKOVITS, R. & CAHALON, L. 2001. Dopamine interacts directly with its D3 and D2 receptors on normal human T cells, and activates betal integrin function. Eur J Immunol, 31, 3504-12.

LI, J., ZHANG, Z., LIU, X., WANG, Y., MAO, F., MAO, J., LU, X., JIANG, D., WAN, Y., LV, J. Y., CAO, G., ZHANG, J., ZHAO, N., ATKINSON, M., GREINER, D. L., PRUD′HOMME, G. J., JIAO, Z., LI, Y. & WANG, Q. 2015. Study of GABA in Healthy Volunteers: Pharmacokinetics and Pharmacodynamics. Front Pharmacol, 6, 260.

MARSHALL, F. H., JONES, K. A., KAUPMANN, K. & BETTLER, B. 1999. GABAB receptors—the first 7TM heterodimers. Trends Pharmacol Sci, 20, 396-9.

MENDU, S. K., AKESSON, L., JIN, Z., EDLUND, A., CILIO, C., LERNMARK, A. & BIRNIR, B. 2011. Increased GABAA channel subunits expression in CD8+ but not in CD4+ T cells in BB rats developing diabetes compared to their congenic littermates. Mol Immunol, 48, 399-407.

MENDU, S. K., BHANDAGE, A., JIN, Z. & BIRNIR, B. 2012. Different subtypes of GABAA receptors are expressed in human, mouse and rat T lymphocytes. PLoS One, 7, e42959.

OLSEN, R. W. & SIEGHART, W. 2008. International Union of Pharmacology. LXX. Subtypes of gamma-aminobutyric acidA receptors: classification on the basis of subunit composition, pharmacology, and function. Update. Pharmacol Rev, 60, 243-60.

OLSEN, R. W. & SIEGHART, W. 2009. GABAA receptors: subtypes provide diversity of function and pharmacology. Neuropharmacology, 56, 141-8.

PAN, Y., RIPPS, H. & QIAN, H. 2006. Random assembly of GABA rhol and rho2 subunits in the formation of heteromeric GABAC receptors. Cell Mol Neurobiol, 26, 289-305.

PETTY, F. & SHERMAN, A. D. 1984. Plasma GABA levels in psychiatric illness. J Affect Disord, 6, 131-8.

PICELLI, S., BJORKLUND, A. K., FARIDANI, O. R., SAGASSER, S., WINBERG, G. & SANDBERG, R. 2013. Smart-seq2 for sensitive full-length transcriptome profiling in single cells. Nat Methods, 10, 1096-8.

PICELLI, S., BJORKLUND, A. K., REINIUS, B., SAGASSER, S., WINBERG, G. & SANDBERG, R. 2014. Tn5 transposase and tagmentation procedures for massively scaled sequencing projects. Genome Res, 24, 2033-40.

PLOG, B. A. & NEDERGAARD, M. 2018. The Glymphatic System in Central Nervous System Health and Disease: Past, Present, and Future. Annu Rev Pathol., 13, 379-94.

RAMSKOLD, D., WANG, E. T., BURGE, C. B. & SANDBERG, R. 2009. An abundance of ubiquitously expressed genes revealed by tissue transcriptome sequence data. PLoS Comput Biol, 5, e1000598.

REYES-GARCIA, M. G., HERNANDEZ-HERNANDEZ, F., HERNANDEZ-TELLEZ, B. & GARCIA-TAMAYO, F. 2007. GABAA receptor subunits RNA expression in mice peritoneal macrophages modulate their IL-6/IL-12 production. J Neuroimmunol, 188, 64-8.

RING, H., MENDU, S. K., SHIRAZI-FARD, S., BIRNIR, B. & HALLBOOK, F. 2012. GABA maintains the proliferation of progenitors in the developing chick ciliary marginal zone and non-pigmented ciliary epithelium. PLoS One, 7, e36874.

SCHMITTGEN, T. D. & LIVAK, K. J. 2008. Analyzing real-time PCR data by the comparative C(T) method. Nat Protoc, 3, 1101-8.

TIAN, J., CHAU, C., HALES, T. G. & KAUFMAN, D. L. 1999. GABAA receptors mediate inhibition of T cell responses. J Neuroimmunol, 96, 21-8.

TIAN, J., DANG, H., CHEN, Z., GUAN, A., JIN, Y., ATKINSON, M. A. & KAUFMAN, D. L. 2013. gamma-Aminobutyric acid regulates both the survival and replication of human beta-cells. Diabetes, 62, 3760-5.

TIAN, J., LU, Y., ZHANG, H., CHAU, C. H., DANG, H. N. & KAUFMAN, D. L. 2004. Gamma-aminobutyric acid inhibits T cell autoimmunity and the development of inflammatory responses in a mouse type 1 diabetes model. J Immunol, 173, 5298-304.

TONG, Y., LI, Z., ZHANG, H., XIA, L., ZHANG, M., XU, Y., WANG, Z., DEEM, M. W., SUN, X. & HE, J. 2016. T Cell Repertoire Diversity Is Decreased in Type 1 Diabetes Subjects. Genomics Proteomics Bioinformatics, 14, 338-348.

WANG, T. L., GUGGINO, W. B. & CUTTING, G. R. 1994. A novel gamma-aminobutyric acid receptor subunit (rho 2) cloned from human retina forms bicucullineinsensitive homooligomeric receptors in Xenopus oocytes. J Neurosci, 14, 6524-31.

1. A method for identifying a subject at risk of developing an autoimmune or inflammatory disorder, comprising isolating Peripheral Blood Mononuclear Cells (PBMCs) from a blood sample obtained from said subject;
a) culturing a subset of said PBMCs in the presence of GABA, or a GABA receptor agonist;
b) culturing a subset of said PBMCs in the absence of GABA, or a GABA receptor agonist;
c) measuring the proliferation of said PBMCs in the presence and absence of GABA or GABA receptor agonist;
wherein a reduced proliferation in the presence of GABA or GABA receptor agonist relative the proliferation in the absence of GABA or GABA receptor agonist is indicative of the subject being at risk of developing an autoimmune or inflammatory disorder.

2. A method for identifying a subject at risk of developing an autoimmune or inflammatory disorder, comprising isolating Peripheral Blood Mononuclear Cells (PBMCs) from a blood sample obtained from said subject;
a) culturing a subset of said PBMCs in the presence of GABA, or a GABA receptor agonist;
b) culturing a subset of said PBMCs in the absence of GABA, or a GABA receptor agonist;
c) obtaining a cytokine profile of said PBMCs in the presence and absence of GABA or GABA receptor agonist;
wherein a change in the cytokine profile in the presence of GABA or GABA receptor agonist relative the cytokine profile in the absence of GABA or GABA receptor agonist is indicative of the subject being at risk of developing an autoimmune or inflammatory disorder.

3. The method according to claim 2, wherein the change in the cytokine profile is a significant decrease of the expression of CDCP1 and TNF in the presence of GABA or GABA receptor agonist relative the expression in the absence of GABA or GABA receptor agonist.

4. A method of prevention of development of an autoimmune or inflammatory disorder, comprising administering GABA, or a GABA receptor agonist, to a subject identified to be at risk according to the method of claim 1.

5. A method for assessing a subject's susceptibility to treatment with gamma-aminobutyric acid (GABA) or a GABA receptor agonist, comprising isolating Peripheral Blood Mononuclear Cells (PBMCs) from a blood sample obtained from said subject;
a) culturing a subset of said PBMCs in the presence of GABA, or a GABA receptor agonist;
b) culturing a subset of said PBMCs in the absence of GABA, or a GABA receptor agonist;
c) measuring the proliferation of said PBMCs in the presence and absence of GABA or GABA receptor agonist;
wherein a reduced proliferation in the presence of GABA or GABA receptor agonist relative the proliferation in the absence of GABA or GABA receptor agonist is indicative of the subject being susceptible to treatment with GABA or a GABA receptor agonist.

6. The method according to claim 5, wherein a statistically significant reduction of proliferation in the presence of GABA or GABA receptor agonist relative the proliferation in the absence of GABA or GABA receptor agonist, such as a reduction by 10, 20, 30, 40, 50, 60, 70, 80, or 90%, is indicative of the subject being susceptible to treatment with GABA.

7. A method for assessing a subject's susceptibility to treatment with gamma-aminobutyric acid (GABA) or a GABA receptor agonist, comprising isolating Peripheral Blood Mononuclear Cells (PBMCs) from a blood sample obtained from said subject;
a) culturing a subset of said PBMCs in the presence of GABA, or a GABA receptor agonist;
b) culturing a subset of said PBMCs in the absence of GABA, or a GABA receptor agonist;
c) obtaining a cytokine profile of said PBMCs in the presence and absence of GABA or GABA receptor agonist;
wherein a change in the cytokine profile in the presence of GABA or GABA receptor agonist relative the cytokine profile in the absence of GABA or GABA receptor agonist is indicative of the subject being susceptible to treatment with GABA or a GABA receptor agonist.

8. The method according to claim 7, wherein the change in the cytokine profile is a significant decrease of the expression of CDCP1 and TNF in the presence of GABA or GABA receptor agonist relative the expression in the absence of GABA or GABA receptor agonist.

9. The method according claim 5, wherein the subject suffers from an autoimmune or inflammatory disorder.

10. The method according to claim 1, wherein CD4⁺ cells are isolated from said PBMCs and used in the steps a) and b).

11. A method for treatment comprising assessing a subject's susceptibility to treatment with gamma-aminobutyric acid (GABA) or a GABA receptor agonist, comprising performing the method according to claim 5, and administering GABA or a GABA receptor agonist to said subject only if the subject is indicated as susceptible to treatment with GABA or a GABA receptor agonist.

12. The method according to claim 4, wherein the method is for treatment of an autoimmune or inflammatory disorder, said disorder being chosen from the group consisting of Type 1 Diabetes, presymptomatic Type 1 diabetes of stage 1, presymptomatic Type 1 diabetes of stage 2 allergy, Grave's disease, Hashimoto's thyroiditis, hypoglyceimia, multiple sclerosis, mixed essential cryoglobulinemia, systemic lupus erthematosus, Rheumatoid Arthritis (RA), Coeliac disease, or any combination thereof.

13. The method according to claim 12, wherein the disorder is selected from the group consisting of Type 1 Diabetes.

14. The method according to claim 4, further comprising administering a Positive Allosteric Modulator of a GABAA receptor (PAM) wherein said PAM is selected from the group consisting of allobarbital (5,5-diallylbarbiturate), amobarbital (5-ethyl-5-isopentyl-barbiturate), aprobarbital (5-allyl-5-isopropyl-barbiturate), alphenal (5-allyl-5-phenyl-barbiturate), barbital (5,5-diethylbarbiturate), brallobarbital (5-allyl-5-(2-bromo-allyl)-barbiturate), pentobarbital (5-ethyl-5-(1-methylbutyl)-barbiturate), phenobarbital (5-ethyl-5-phenylbarbiturate), secobarbital (5-[(2R)-pentan-2-yl]-5-prop-2-enyl-barbiturate), alprazolam, bromazepam, chlordiazepoxide, midazolam, clonazepam, clorazepate, diazepam, estazolam, flurazepam, halazepam, ketazolam, lorazepam, nitrazepam, oxazepam, prazepam, quazepam, temazepam, and triazolam.

15. The method according to claim 4, wherein GABA, and optionally a PAM, is administered in an amount effective to inhibit a Th2-type of response for the subject.

16. The method according to claim 4, wherein GABA, and optionally a PAM, is administered in an amount effective induce a T-regulatory response for the subject.

17. The method according to claim 16, whereby the T-regulatory response is measured as an increase in IL-4 secretion following GABA treatment.

18. The method according to claim 4, wherein GABA, and optionally a PAM is administered in an amount effective to inhibit a Th2 type and a Th1 type of response for the subject.

19-21. (canceled)

22. A method of prevention of development of an autoimmune or inflammatory disorder, comprising administering GABA, or a GABA receptor agonist, to a subject identified to be at risk according to the method of claim 2.
